(19) United States
(12) Patent Application Publication
(10) Pub. No.: US 2012/0172380 A1
(43) Pub. Date: Jul. 5, 2012

(54) NOVEL 4-(TETRAZOL-5-YL)-QUINAZOLINE DERIVATIVES AS ANTI CANCER AGENT

(75) Inventors: Durga prasad Konakanchi, Hyderabad (IN); Subba Rao Pula, Hyderabad (IN); Lakshmi Ananthaneni, Hyderabad (IN); Pulla Reddy Muddasani, Hyderabad (IN); Kali Satya Bhujanga Rao Adibhatla, Hyderabad (IN); Venkaiah Chowdary Nannapaneni, Hyderabad (IN)

(73) Assignee: Natco Pharma Limited, Hyderabad (IN)

(21) Appl. No.: 13/330,304

(22) Filed: Dec. 19, 2011

Related U.S. Application Data
(63) Continuation-in-part of application No. 12/740,527, filed on Apr. 29, 2010, now Pat. No. 8,080,558.

(30) Foreign Application Priority Data
Oct. 29, 2007 (IN) 2445/CHE/2007

(51) Int. Cl. A61K 31/517; A61P 35/00

(52) U.S. Cl. 514/266.23

(57) ABSTRACT

The invention relates to substituted 4-(tetrazol-5-yl)-quinazoline derivatives of the formula-I,

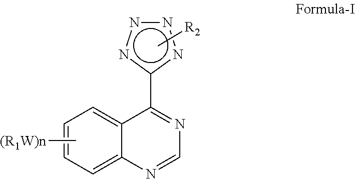

or pharmaceutically acceptable salts thereof, which possess anti-proliferative activity such as anti-cancer activity and are accordingly useful in methods of treatment of the human or animal body. The invention also relates to processes for the manufacture of substituted 4-(tetrazol-5-yl)-quinazoline derivatives, to pharmaceutical compositions containing the compound and to its use in the manufacture of medicaments for the production of an anti-proliferative effect in a warm-blooded animal such as man.

### CROSS REFERENCE TO RELATED APPLICATION

The present application claims priority as a continuation in part to U.S. patent application Ser. No. 12/740,527, filed Apr. 29, 2010, and to issue as U.S. Pat. No. 8,080,558, on Dec. 20, 2011,which is a National Stage Application of PCT/IN2008/000708, filed 28 Oct. 2008, which claims benefit of Ser. No. 2445/CHE/2007, filed 29 Oct. 2007 in India and which applications are incorporated herein by reference. A claim of priority to all, to the extent appropriate is made.

### FIELD OF INVENTION

The invention relates to substituted 4-(tetrazol-5-yl)-quinazoline derivatives of the formula-I,

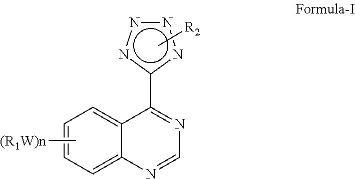

or pharmaceutically-acceptable salts thereof, which possess anti-proliferative activity such as anti-cancer activity and are accordingly useful in methods of treatment of the human or animal body. The invention also relates to processes for the manufacture of substituted 4-(tetrazol-5-yl)-quinazoline derivatives, to pharmaceutical compositions containing the compound and to its use in the manufacture of medicaments for the production of an anti-proliferative effect in a warm-blooded animal such as man.

### BACKGROUND OF THE INVENTION

Many of the earlier treatment regimes for cell proliferation diseases such as psoriasis and cancer utilize compounds, which inhibit DNA synthesis. Such compounds are toxic to cells generally but their toxic effect on rapidly dividing cells such as tumor cells can be beneficial. Alternative approaches to anti-proliferative agents which act by mechanisms other than the inhibition of DNA synthesis have the potential to display enhanced selectivity of action.

In recent years it has been discovered that a cell may become cancerous by virtue of the transformation of a portion of its DNA into an oncogene i.e. a gene which, on activation, leads to the formation of malignant tumor cells. Several such oncogenes give rise to the production of peptides, which are receptors for growth factors. The growth factor receptor complex subsequently leads to an increase in cell proliferation. It is known, for example, that several oncogenes encode tyrosine Kinase enzymes and that certain growth factor receptors are also tyrosine Kinase enzymes.

The epidermal growth factor receptor (EGFR) pathway has been implicated in tumor-promoting events such as cell division, cell adhesion and migration, angiogenesis, and anti-apoptosis. EGFR belongs to the erbB family of structurally related receptors, including EGFR (HER-1, erbB1), HER-2/neu (erbB2), HER-3 (erbB3), and HER-4 (erbB4). The central role of EGFR in cancer has engendered efforts to develop EGFR antagonists. The first-generation small-molecule inhibitors act as ATP analogs competing reversibly for the TK catalytic site. Newer inhibitors that are under development produce irreversible antagonism and/or target multiple erbB receptors

Receptor tyrosine kinases are active in the transmission of biochemical signals, which initiate cell replication. Class I receptor tyrosine kinases include the EGF family of receptor tyrosine kinases such as the EGF, TGFα, NEU, erbB, Xmrk, HER and let23 receptors, Class II receptor tyrosine kinases include the insulin family of receptor tyrosine kinases such as the insulin, IGFI and insulin-related receptor (IRR) receptors and Class III receptor tyrosine kinases include the platelet-derived growth factor (PDGF) family of receptor tyrosine kinases such as the PDGFα, PDGFβ. and colony stimulating factor 1 (CDF1) receptors.

It is known that Class I kinases such as the EGF family of receptor tyrosine kinases are frequently present in common human cancers such as breast cancer, non-small cell lung cancers (NSCLCs) including adenocarcinomas, squamous cell cancer of the lung, esophageal cancer; gastrointestinal cancer such as colon, rectal or stomach cancer; cancer of the prostate, leukemia, and ovarian, bronchial or pancreatic cancer. It is also known that EGF type tyrosine Kinase activity is rarely detected in normal cells whereas it is more frequently detectable in malignant cells. It has been shown more recently that EGF receptors which possess tyrosine kinase activity are over expressed in many human cancers such as brain, lung squamous cell, bladder, gastric, breast, head and neck, esophageal, gynecological and thyroid tumors. Accordingly it has been recognized that an inhibitor of receptor tyrosine kinases should be of value as a selective inhibitor of the growth of mammalian cancer cells.

Applications Nos. EP0520722, EP0566226 and EP0635498 disclose that certain quinazoline derivatives which bear an anilino substituent at the 4-position possess receptor tyrosine kinase inhibitory activity. Application No. EP0602851 discloses that certain quinazoline derivatives which bear a heteroarylamino substituent at the 4-position also possess receptor tyrosine kinase inhibitory activity.

Patent Application No. WO 92/20642 discloses that certain aryl and heteroaryl compounds inhibit EGF and/or PDGF receptor tyrosine kinase. There is the disclosure of certain quinazoline derivatives therein but no mention is made of 4-anilinoquinazoline derivatives.

The in vitro anti-proliferative effect of a 4-anilinoquinazoline derivative has been disclosed by Fry et al., Science, 1994, 265, 1093. It was stated that the compound 4-(3′-bromoanilino)-6,7-dimethoxyquinazoline was a highly potent inhibitor of EGF receptor tyrosine kinase.

AstraZeneca has developed and launched gefitinib (U.S. Pat. No. 5,770,599), of the formula II,

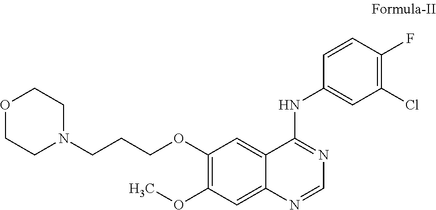

an orally active, selective epidermal growth factor receptor-tyrosine kinase inhibitor (EGFR-TK1). It is indicated as monotherapy for the continued treatment of patients with locally advanced or metastatic non-small cell lung cancer after failure of both platinum-based and docetaxel chemotherapies that are benefiting or have benefited from gefitinib. The brand name is Iressa.

OSI Pharmaceuticals has developed and launched Erlotinib (U.S. Pat. No. 5,747,498) of formula-III,

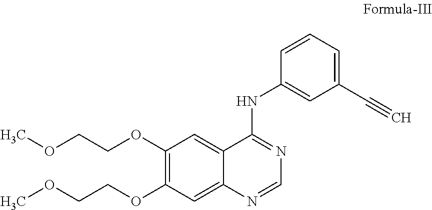

an orally active, ATP-competitive small-molecule inhibitor of EGFR TK. It is presently being used as a standard treatment for non-small cell lung cancer (NSCLC) and pancreatic cancer. Its activity is expected to be enhanced when combined with standard cytotoxic antibiotic anti-cancer drugs. The brand name is Tarceva.

Recently mutations in the ATP binding pocket of the intracellular catalytic domain of the EGF receptor have been discovered in certain sub-sets of non-small cell lung cancers (NSCLCs). The presence of mutations in the receptor appear to correlate with response to EGFR tyrosine kinase inhibitors such as gefitinib, although it is becoming evident that the clinical benefits of compounds such as gefitinib and erlotinib are not likely to be mediated by EGFR mutations alone. It has been demonstrated that ligand stimulation results in a different phosphorylation pattern in mutated receptors compared with that seen in wild-type receptors and it is thought that mutant EGF receptors selectively transduce survival signals on which NSCLCs become dependent. Inhibition of those signals by compounds such as gefitinib may contribute to the efficacy of such drugs. Similarly, mutations within the erbB2 kinase domain have recently been discovered in certain primary tumors, such as NSCLC, glioblastoma and gastric and ovarian tumors. Accordingly the inhibition of the EGF and/or erbB2 tyrosine kinase in both wild-type and mutated receptors is an important target that would be expected to provide an anti-cancer effect.

Amplification and/or activity of members of the erbB type receptor tyrosine kinases have been detected and so have been implicated to play a role in a number of non-malignant proliferative disorders such as psoriasis, benign prostatic hyperplasia (BPH), atherosclerosis and restenosis. WO 96/09294, WO 96/15118, WO 96/16960, WO 96/30347, WO 96/33977, WO96/33978, WO 96/33979, WO 96/33980, WO 96/33981, WO 97/03069, WO 97/13771, WO 97/30034, WO 97/30035, WO 97/38983, WO 98/02437, WO 98/02434, WO 98/02438, WO 98/13354, WO 99/35146, WO 01/21596, WO 01/55141 and WO 02/18372 each disclose that certain quinazoline derivatives which bear an anilino substituent at the 4-position possess receptor tyrosine kinase inhibitory activity. WO 99/35132 discloses certain 4-(indazol-5-ylamino)quinazoline derivatives. However, none of these quinazoline derivatives contain a substituent at the 5-position on the quinazoline ring.

WO 01/94341 discloses that certain quinazoline derivatives which carry a 5-substituent are inhibitors of the Src family of non-receptor tyrosine kinases, such as c-Src, c-Yes and c-Fyn. There is no disclosure on WO 01/94341 of 4-(indazol-5-yl amino)qinazoline derivatives wherein the nitrogen atom of the indazolyl group is substituted by a substituent containing an aryl or a heteroaryl group.

WO 03/040108 and WO 03/040109 each disclose that certain quinazoline derivatives which carry a 5-substituent are inhibitors of the erbB family of tyrosine kinase inhibitors, particularly EGF and erbB2 receptor tyrosine kinases. WO 03/040108 and WO 03/040109 each disclose certain 4-(indazol-5-ylamino)quinazoline derivatives. None of the quinazoline derivatives disclosed contain an acyl amino ethoxy group at the 5-position on the quinazoline ring.

US-2004/0048880 discloses certain 4-anilinoquinazoline derivatives and their use in treating tumoral diseases. The quinazoline derivatives do not contain a substituent at the 5-position on the quinazoline ring. WO 2004/46101 discloses certain 4-(indazol-5-ylamino)quinazoline derivatives and their use as inhibitors of EGF and erbB 2 receptor tyrosine kinases. The quinazoline derivatives do not contain a substituent at the 5-position on the quinazoline ring.

WO 2004/093880 and WO 2005/051923 each disclose certain 4-anilinoquinazoline derivatives and their use as inhibitors of erbB2 receptor tyrosine kinase. Neither of these documents disclose a 4-(indazol-5-ylamino)quinazoline derivative.

There remains a need to find further compounds with good in vivo activity together with improved pharmacological characteristics compared with known erbB tyrosine kinase inhibitors, particularly compounds that are selective erbB2 tyrosine kinase inhibitors.

### SUMMARY OF THE INVENTION

The present invention relates to methods employing substituted 4-(tetrazol-5-yl)-quinazoline derivatives of the formula-I,

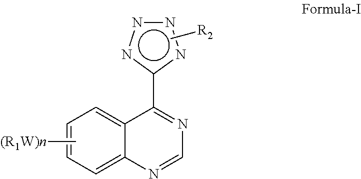

or pharmaceutically-acceptable salts thereof, which compounds are described in detail hereinbelow. These compounds possess anti-proliferative activity such as anti-cancer activity and are accordingly useful in methods of treatment of the human or animal body. The invention also relates to processes for the manufacture of substituted 4-(tetrazol-5-yl)-quinazoline derivatives, to pharmaceutical compositions containing the compound and to its use in the manufacture of medicaments for the production of an anti-proliferative effect in a warm-blooded animal such as man.

The present invention includes a method of treating a hyper proliferative disease in a mammal. This method includes administering to said mammal a therapeutically effective amount of the compound of formula I, which formula is described in detail hereinbelow. The hyper proliferative disorder can be cancer. The cancer can be lung, squamous cell, bladder, gastric, pancreatic, breast, head, neck, esophageal, brain, gynecological or thyroid cancer. The hyperproliferative disease can be noncancerous.

The present compound for formula I can be employed in the manufacture of a medicament for treating the human or animal body.

In an embodiment, the compound of formula I can be:
6,7-Dimethoxy-4-(1-(3-nitrobenzyl)-1H-tetrazol-5-yl)quinazoline;
3-((5-(6,7-dimethoxyquinazolin-4-yl)-1H-tetrazol-1-yl)methyl)aniline;
6,7-Dimethoxy-4-(1-(3-aminobenzyl)-1H-tetrazol-5-yl)quinazoline hydrochloride;
6,7-dimethoxy-4-(1-((l-methyl-1H-imidazol-2-yl)methyl-1H-tetrazol-5-yl)quinazoline;
6,7-dimethoxy-4-(1-(pyridin-2-ylmethyl)-1H-tetrazol-5-yl)-quinazoline;
6,7-diethoxy-4-(1H-tetrazol-5-yl)quinazoline;
6,7-diethoxy-4-(1-(3-nitrobenzyl)-1H-tetrazol-5-yl)quinazoline;
3-((5-(6,7-diethoxyquinazolin-4-yl)-1H-tetrazol-1-yl)methyl)aniline;
6,7-diethoxy-4-(1-((l-methyl-1H-imidazol-2-yl)methyl)-1H-tetrazol-5-yl)quinazoline;
6,7-diethoxy-4-(1-(pyridin-2-ylmethyl)-1H-tetrazol-5-yl)quinazoline;
6,7-dipropoxy-4-(1H-tetrazol-5-yl)quinazoline;
6,7-di-n-propoxy-4-(1-(3-nitrobenzyl)-1H-tetrazol-5-yl)quinazoline;
3-((5-(6,7-di-n-propoxyquinazolin-4-yl)-1H-tetrazol-1-yl)methyl)aniline;
4-(1-((l-methyl-1H-imidazol-2-yl)methyl)-1H-tetrazol-5-yl)6,7-di-n-propoxy quinazoline; or
6,7-di-n-propoxy-4-(1-(pyridin-2-ylmethyl)-1H-tetrazol-5-yl)-quinazoline.

In an embodiment, the compound of formula I can be a compound of Formula IV, of Formula V or a pharmaceutically acceptable salt thereof:

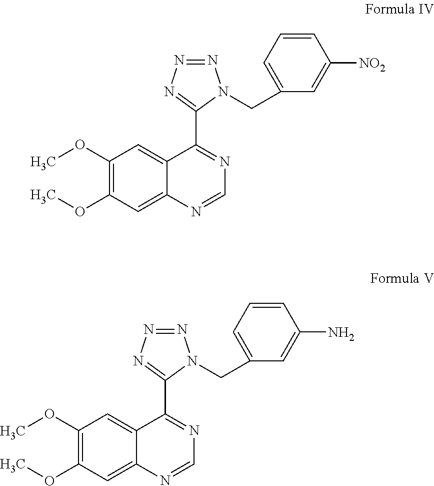

### BRIEF DESCRIPTION OF THE FIGURES

FIG. 1 shows a Western blot analysis of H1299 cells treated with Erlotinib, NRC1005. Erlotinib and Compound V (NRC-1005) caused a dose dependent decrease in EGFR levels, but no significant change in expression levels of PI3K or AKT was observed in any of drug treatments.

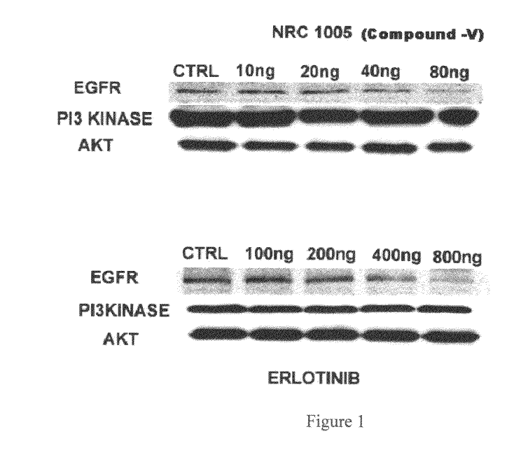

FIG. 2 illustrates results of a matrigel invasion assay of A549 cells treated with Erlotinib, Compound IV (NRC-1004), and Compound V (NRC-1005).

FIG. 3 illustrates results of a matrigel invasion assay of H1299 cells treated with Erlotinib, Compound IV (NRC-1004), and Compound V (NRC-1005).

FIG. 4 illustrates the decrease in tumor size induced by oral administration of Erlotinib, Compound IV (NRC-1004), and Compound V (NRC-1005) in nude mice implanted with A549 human lung tumors.

FIG. 5 illustrates nude mice implanted with A549 luciferase expressing cells and then treated with various concentrations of Erlotinib by oral or ip routes.

FIG. 6 illustrates nude mice implanted with A549 luciferase expressing cells and then treated with various concentrations of Compound V (NRC-1005) by oral or ip routes.

The patent or application file contains at least one drawing executed in color. Copies of this patent or patent publication with color drawing(s) will be provided by the Office upon request and payment of the necessary fee.

### DETAILED DESCRIPTION OF THE INVENTION

Surprisingly, we have now found that a select group of substituted 4-(tetrazol-5-yl)-quinazoline derivatives of the present invention, or a pharmaceutically-acceptable salt thereof, possess potent anti-tumor activity.

The present invention relates to substituted-4-(tetrazol-5-yl)-quinazoline derivatives of formula-I,

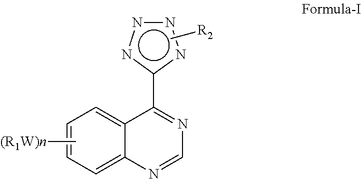

where

n is 1, 2, or 3;

W is selected from a single bond, —O—, —S—, —COR₆, —NH—, —SO—, —SO₂—, —NR₆CO—, —CONR₆—, —SO₂NR₇—, —NR₇SO₂—, or —NR₈— (wherein R₆, R₇ and R₈ each independently represents hydrogen, C₁-C₆alkyl, C₃-C₆cycloalkyl, C₂-C₅alkenyl, C₂-C₅alkynyl, or each R₁ is R₉ where R₉ is independently selected from C₁-C₆ branched alkyl, C₂-C₆ branched alkenyl or C₂-C₆ branched alkynyl ;

or each R₁ is independently selected from the group consisting of hydrogen, halogen, hydroxy, amino, hydroxylamino, carboxy, nitro, guanidino, ureido, cyano, trifluoromethyl, azido;

or each R₁ is independently selected from the group consisting of C₁-C₆alkyl, C₃-C₆ cycloalkyl, aryl, heterocyclyl, R3-substituted aryl, R3-substituted heterocyclyl, aryl C₁-C₆alkoxy, C₃-C₆cycloalkoxy, (C₁-C₆)alkanoyloxy, R₅-aryloxy, C₁-C₆alkoxy C₁-C₆alkyloxy, C₁-C₆alkoxy-C₃-C₆cycloalkyloxy, C₁-C₆alkoxy-R₅-aryloxy, C₁-C₆alkoxy-heterocyclyloxy, C₁-C₆alkoxy-fused-heterocyclyloxy, N-mono(C₁-C₆)alkylamino, N,N-di(C₁-C₆)alkylamino, formamido, amido, acetamido, C₁-C₆-alkoxyamino, hydrazino, trifluoromethoxy, alkenyl, alkynyl, aryl, heterocyclyl, fused aryl, fused heteroaryl and fused heterocyclyl; where R₃ is selected from C₁-C₆ alkyl, C₃-C₆ cycloalkyl, aryl, and aralkyl; R₅ is independently hydrogen or R₄; and where R₄ is C₁-C₄ alkyl;

or each R₁ is independently selected from R₉-substituted by halogen, hydroxy, amino, hydroxylamino, carboxy, nitro, guanidino, ureido, cyano, trifluoromethyl, azido; wherein R₉ is selected from the group consisting of R₄, —OR₅, —NR₅R₅, —C(O)R₆, —NHOR₄, —OC(O)R₅, P and -QR₄; R₆ is R₃, —OR₅ or —NR₅R₅; P is selected from piperidino, morpholino, pyrrolidino, 4-R₃-piperazin-1-yl, imidazol-1-yl, 4-pyridon-1-yl, —(C₁-C₄alkylene)(CO₂H), phenoxy, phenyl, phenylsulfonyl, C₂-C₄alkenyl, and —(C₁-C₄alkylene)C(O)NR₅ R₅ ; and Q is S, SO, or SO₂;

or each R₁ is independently selected from phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R₄—(C₂-C₄)-alkanoylamino and wherein said —NHSO₂ R₄, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R₄—(C₂-C₄)-alkanoylamino R₁ groups are optionally substituted by 1 or 2 substituents independently selected from halo, C₁-C₄alkyl, cyano, methanesulfonyl and C₁-C₄alkoxy;

R₂ is hydrogen or selected from the group consisting of C₁-C₆alkyl, C₃-C₆cycloalkyl, (C₁-C₆)carbonyloxyalkyl, R₄-aryl, R₄-aryl substituted with (R₁ᵢ)m, where m=1, 2 or 3 and R₁₁ is independently selected from the group consisting of hydrogen, halogen, hydroxy, hydroxylamino, carboxy, nitro, guanidino, ureido, cyano, trifluoromethyl, azido, or R₃(as defined above), R₄-fused aryl, R₄-fused aryl substituted with (R₁₀)m, R₄-heterocyclyl, R₄-heterocyclyl substituted with (R₁₁)m, R₄-fused heterocyclyl, R₄-fused heterocyclyl substituted with (R₁₁)m, R₄-C₁-C₆alkyloxy, R₄-C₁-C₆alkyloxy substituted with (R₁₁)m, R₄-C₃-C₆cycloalkyloxy, R₄-C₃-C₆cycloalkyloxy substituted with (R₁₁)m, C₁-C₆alkoxy-R₅-aryloxy, C₁-C₆alkoxy-R₅-aryloxy substituted with (R₁₁)m, C₁-C₆alkoxy hetero-cyclyloxy, C₁-C₆alkoxy-heterocyclyloxy substituted with (R₁₁)m, C₁-C₆alkoxy fused heterocyclyloxy, C₁-C₆alkoxy fused heterocyclyloxy substituted with (R₁₁)m, N-mono(C₁-C₆)alkylamino, N-mono(C₁-C₆)alkylamino substituted with (R₁₁)m, N,N-di(C₁-C₆)alkylamino, N,N-di(C₁-C₆)alkylamino substituted with (R₁₁)m, formamido, amido, acetamido, C₁-C₆alkoxyamino, hydrazino, trifluoromethoxy, C₂-C₆alkenyl, C₂-C₆alkenyl substituted with (R₁₁)m, C₂-C₆alkynyl, C₂-C₆alkynyl substituted with (R₁₁)m.

Formula-I compounds and pharmaceutically acceptable salts thereof may be prepared by any process known to be applicable to the chemically related compounds. In general, the active compounds may be made from the appropriate substituted 4-halo quinazoline compounds derived from the predecessors substituted 4H-quinazolin-4-ones. The active compounds of present invention can be prepared by the following synthetic Scheme-I.

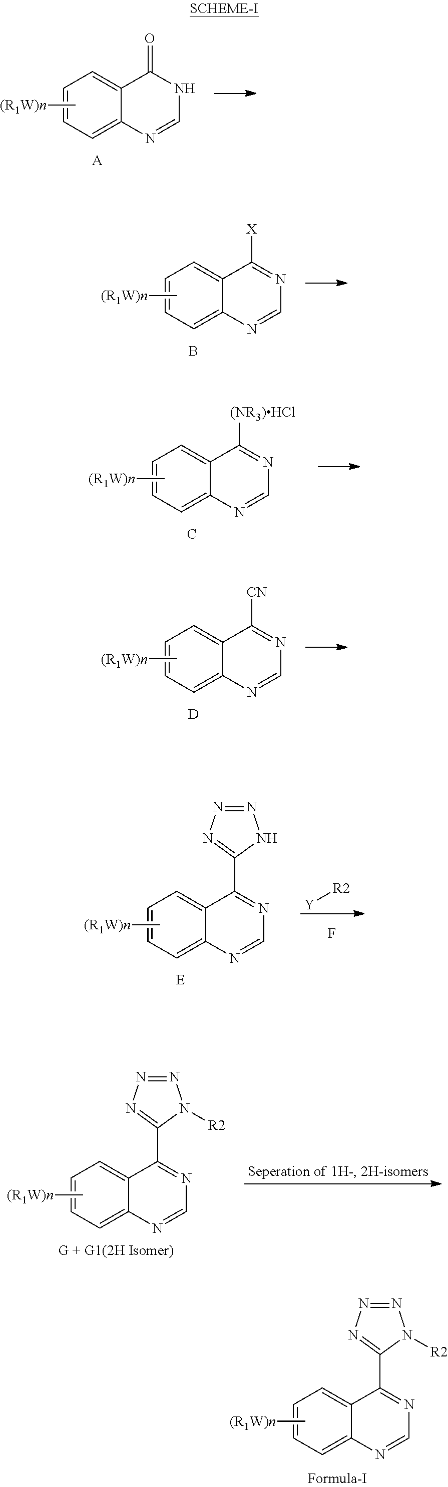

R₁, R₂, R₃ and W are defined as above.

Where X is a halogen or sulfonyl-leaving group.

Where Y is a halogen or sulfonyl-leaving group.

Various compounds of formula-I can be prepared by:

(a) Treating a compound of formula A

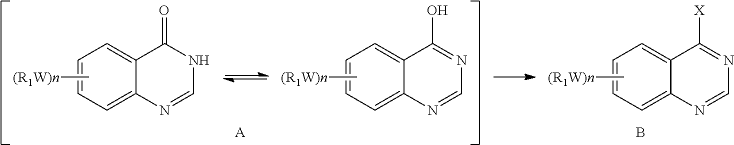

with a halogenating agent such as thionyl halide, phosphorus trihalide, phosphorus pentahalide, phosphoryl trihalide to obtain 4-halo substituted quinazoline derivatives of formula-B, wherein R₁, W and n are as defined above. The reaction can be performed either neatly without any solvent or with solvent such as methylene chloride, ethylene dichloride, toluene, xylene, or cyclohexane, etc. The temperature of the reaction is maintained between 25° C. to 150° C., for example, at the reflux temperature of the halogenating reagent.

(b) Treating the compound of formula-B,

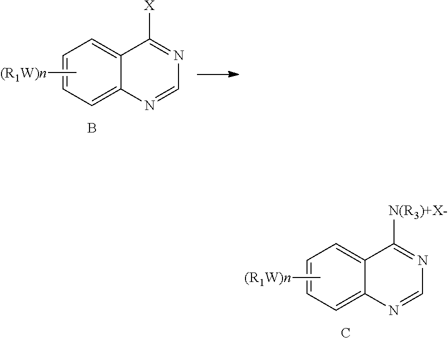

with trialkyl amine (NR₃) (where R₃ is defined as above) in a suitable solvent such as toluene, xylene, cyclohexane or C₁-C₆ linear or branched alkenes to obtain the substituted quinazolinyl-4-trialkylamine halide quaternary salts. The temperature of the reaction is maintained between 25° C. to 150° C., for example at room temperature.

(c) Treating the compound of formula-C,

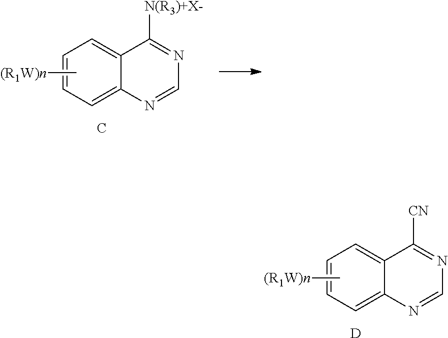

with cyanating agents such as sodium cyanide, potassium cyanide, cuprous cyanide, or trialkyl silyl cyanide etc., in a suitable solvent such as toluene, xylene, cyclohexane or C₁-C₆ linear or branched alkene, dimethylformamide, dimethylacetamide, or formamide, etc., to obtain the substituted 4-amino quinazolines of formula-D, where R₁ and n are as defined above. The temperature of the reaction is maintained between 25° C. to 150° C., for example, at 100° C.-125° C.

(d) Treating the compound of formula-D,

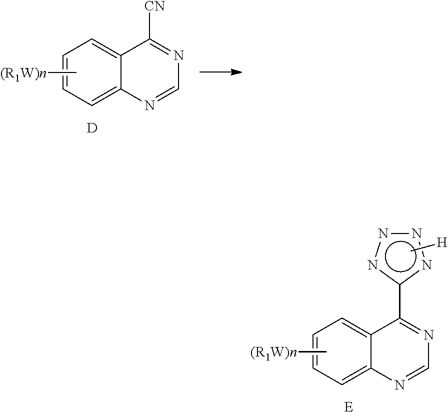

with azidating agents such as sodium azide, or trialkyl silylazide, etc., to obtain the compounds of formula E where R₁ and n are as defined above.

In an embodiment, the reaction is carried out in the presence of a suitable solvent or diluent, for example an alkanol such as methanol, ethanol, isopropanol, an ester solvent such as ethyl acetate, a halogenated solvent such as methylene chloride, chloroform or carbon tetrachloride, an ether solvent such as tetrahydrofuran, 1,4-dioxane, an aromatic hydrocarbon solvent such as toluene, or a dipolar aprotic solvent such as N,N-dimethylformamide, N,N-dimethylacetamide, N-methylpyrrolidin-2-one or dimethyl sulphoxide.

The reaction can be conveniently carried out at a temperature in the range, 10-150° C., e.g., in the range 50-120° C.

(e) Treating the compound of formula-E,

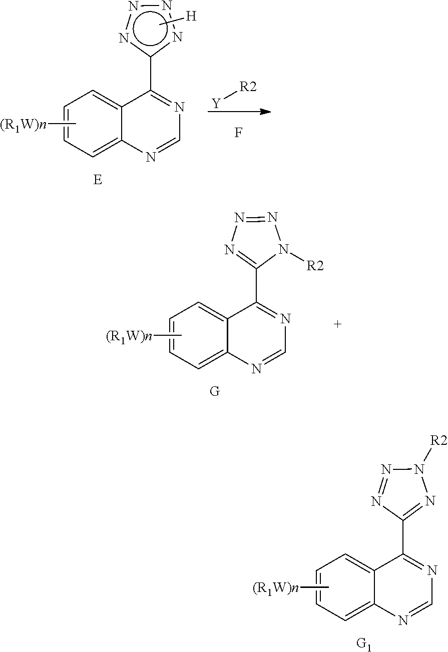

with alkylating agents of formula F (Y and R₂, as defined above) using a base such as alkaline metal carbonates, hydroxides, metal hydrides, metal alkoxides, tetra-alkyl guanidines, alkyl lithium, or LDA, etc. The solvents used are acetonitrile, dimethyl-formamide, dimethylacetamide, tetrahydrofuran, or toluene, etc. The reaction is conveniently carried out at a temperature in the range, for example, 10-150° C., e.g., in the range 20-80° C.

(f) Purifying the compound mixture of formula-G (and its isomer G₁),

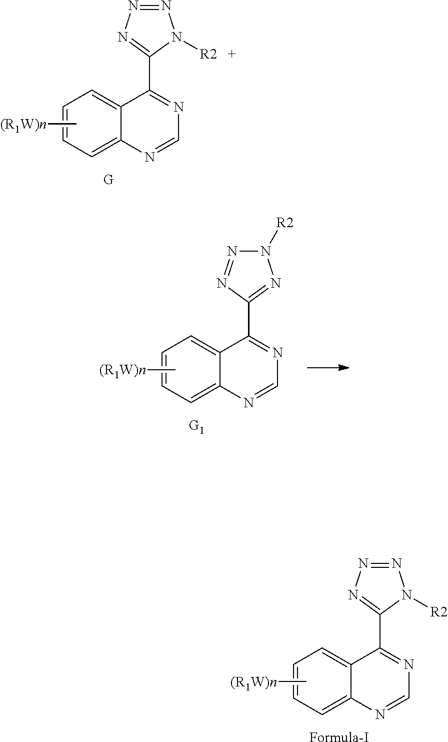

by recrystallisation from a suitable solvent or by preparative chromatography to obtain the required 1 H-tetrazolyl derivative.

Compounds of formula-I with substitutions on 6,7-positions with oxygen linkage and their pharmaceutically acceptable salts there of may be prepared by any process known to be applicable to the chemically related compounds. In general the active compounds may be made from the appropriate substituted 4-chloro-6,7-O-protected quinazoline compounds derived from the predecessors, substituted 4H-quinazolin-4-ones. The active compounds of formula-I are prepared by the following synthetic scheme-II.

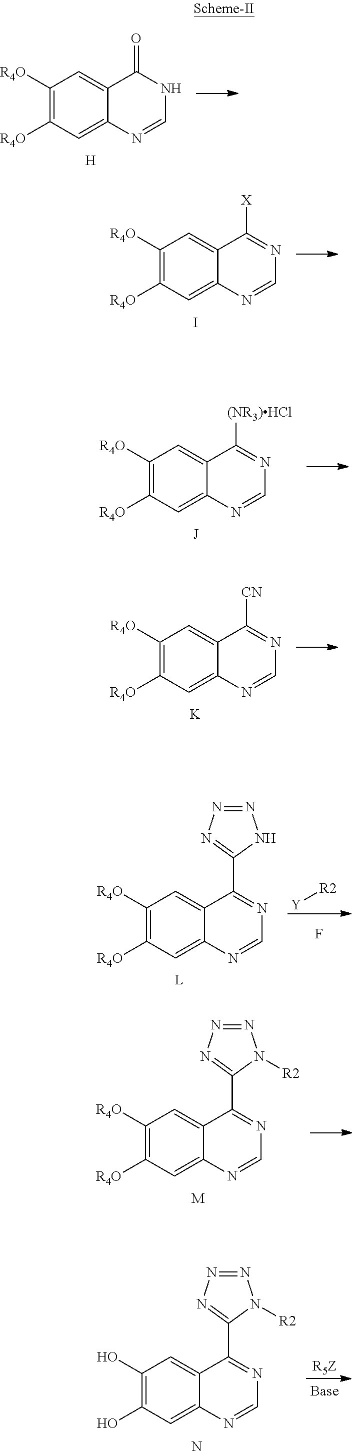

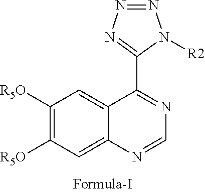

Wherein R₄ and R₅ are defined as above and Y is a suitable protecting group and such as acyl, benzyl, benzoyl, silyl, alkylsulfonyl, arylsulfonyl, or arlkylsufonyl, etc.; Z is halo or a suitable sulfone containing leaving group.

The base used in the O-alkylation step can be an alkali carbonate, alkali hydroxide, metallic alkoxide, alkali hydride, alkyl lithium, tetramethyl guanidine, or the like.

(a) Treating a compound of formula H (or its tautomer of formula H₁)

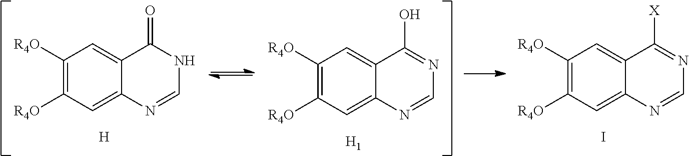

with a halogenating agent such as thionyl halide, phosphorus trihalide, phosphorus pentahalide, phosphoryl trihalide to obtain 4-halo substituted quinazoline derivatives of formula-B, wherein R₄ and X are as defined above. The reaction is tried either neatly without any solvent or with solvent such as methylene chloride, ethylene dichloride, toluene, xylene, or cyclohexane, etc. The temperature of the reaction is maintained between 25° C.-150° C., for example, at the reflux temperature of the halogenating agent.

(b) Treating the compound of formula-I

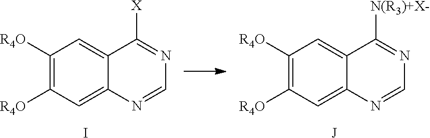

with trialkyl amine (NR₃, where R₃ is defined as above). The reaction is carried out in a suitable solvent such as toluene, xylene, cyclohexane or C₁-C₆ linear or branched alkenes to obtain the substituted quinazolin-4-yl-quaternary trialkylamine halide salts. The temperature of the reaction is maintained between 25° C. to 150° C., for example, at room temperature.

(c) Treating the compound of formula-J,

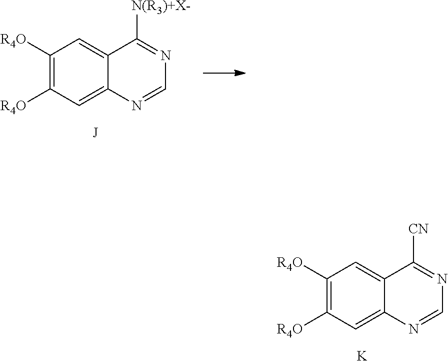

with cyanating agents such as sodium cyanide, potassium cyanide, cuprous cyanide, or trialkyl silyl cyanide etc., in a suitable solvent such as toluene, xylene, cyclohexane, C₁-C₆ linear or branched alkenes, dimethylformamide, dimethylacetamide, or formamide, etc., to obtain the substituted 4-cyanoquinazolines of formula-K, where R₃, R₄ and X are defined as above. The temperature of the reaction is maintained between 25° C. to 150° C., for example, at 100° C.-125° C.

(d) Treating the compound of form

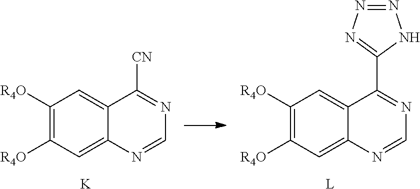

with azidating agents such sodium azide, potassium azide, or trialkyl silyl azide, etc.

In an embodiment, the reaction is carried out in the presence of a suitable solvent or diluent, for example an alkanol such as methanol, ethanol, isopropanol or ester such as ethyl acetate, a halogenated solvent such as methylene chloride, chloroform or carbon tetrachloride, an ether solvent such as tetrahydrofuran or 1,4-dioxane, an aromatic hydrocarbon solvent such as toluene, or a dipolar aprotic solvent such as N,N-dimethylformamide, N,N-dimethylacetamide, N-methylpyrrolidin-2-one or dimethyl sulphoxide.

In an embodiment, the reaction can be carried out at a temperature in the range, for example, 10 to 150° C., e.g., in the range 50 to 120° C.

(e) Treating the compound of formula-L,

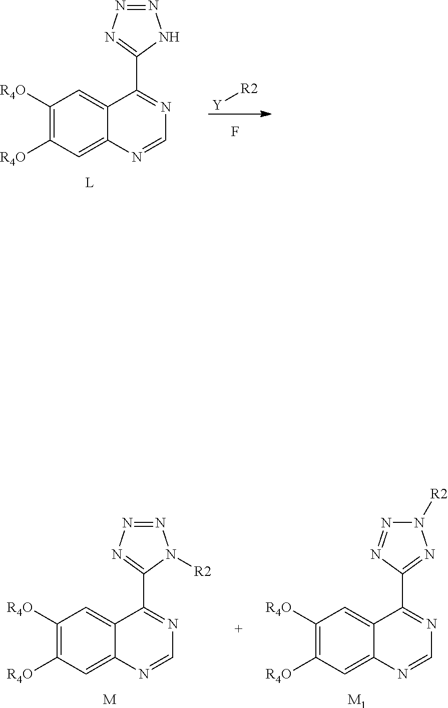

with alkylating agents of compounds of formula F (Y and R₂, as defined above) using a base such as alkaline metal carbonates, hydroxides, hydrides, tetra-alkyl guanidines, alkyl lithium, or LDA, etc. The solvents used are acetonitrile, dimethylformamide, dimethyl acetamide, tetrahydrofuran, or toluene, etc. The reaction is conveniently carried out at a temperature in the range, for example, 10-150° C., e.g., in the range of 20-80° C.

(f) Purifying the compound mixture of formula-M (and its isomer M1),

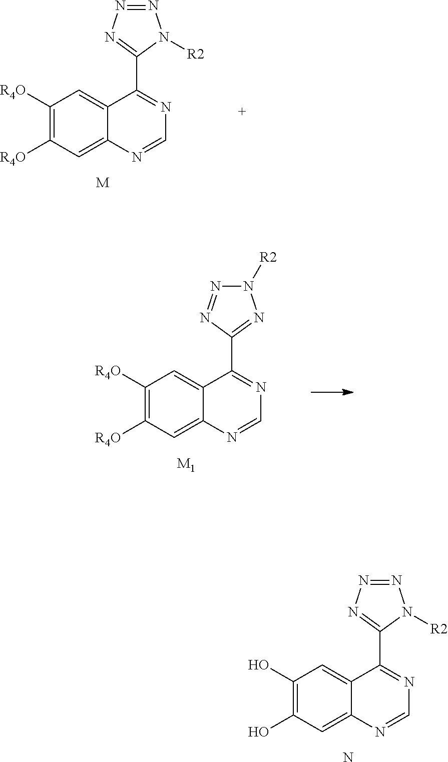

by recrystallization from a suitable solvent or by preparative chromatography to obtain 1H tetrazolyl derivative of formula-N

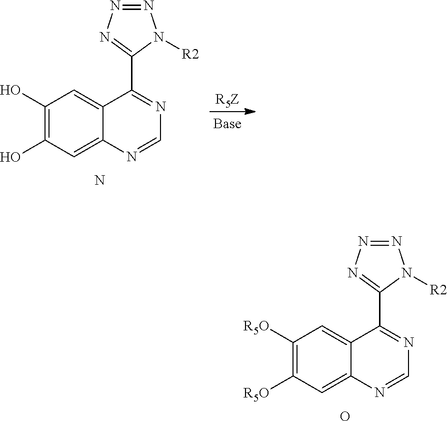

(g) reaction of compounds of formula-N with alkylating agents of formula —R₅Z (where Z and R₅ are as defined above), using a base such as alkaline metal carbonates, hydroxides, metal hydrides, tetra-alkyl guanidines, alkyl lithium, or LDA, etc. The solvent can be acetonitrile, dimethylformamide, dimethyl acetamide, tetrahydrofuran, or toluene, etc. The reaction is conveniently carried out at a temperature in the range of, for example, 10-150° C., e.g., in the range of 20-80° C.

It is also to be understood that certain quinazoline derivatives of the formula I can exist in solvated as well as unsolvated forms such as, for example, hydrated forms. It is to be understood that the invention encompasses all such solvated forms, which possess anti-proliferative activity.

A suitable pharmaceutically acceptable salt of the quinazoline derivative of the invention is, for example, a mono- or di-acid-addition salt of the quinazoline derivative of the invention which is sufficiently basic, for example, an acid-addition salt with, for example, an inorganic or organic acid, for example, hydrochloric, hydrobromic, sulphuric, phosphoric, trifluoroacetic, citric, maleic, tartaric, fumaric, methane-sulphonic, or 4-toluenesulphonic acid.

The invention most particularly relates to novel intermediate compounds of the formula I selected from the group consisting of 6,7-Dimethoxy substituted 4-(tetrazol-5-yl)quinazoline derivatives of formula-IV to VII:

| Compound | | |
|---|---|---|
| Number | Structure | Chemical name |
| IV | 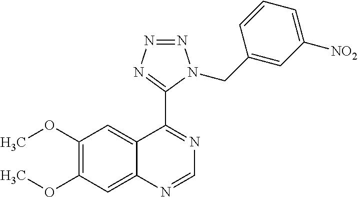 | 6,7-Dimethoxy-4-(1-(3-nitrobenzyl)- 1H-tetrazol-5-yl)quinazoline |
| V | 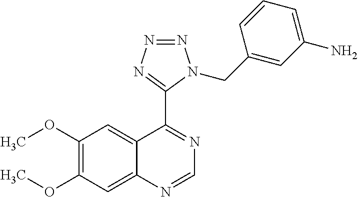 | 6,7-Dimethoxy-4-(1-(3- aminobenzyl)-1H-tetrazol-5-yl) quinazoline |
| VI | 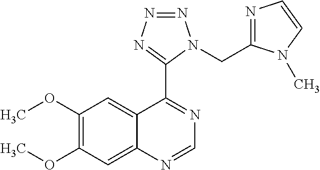 | 6,7-dimethoxy-4-(1-((1-methyl-1H- imidazol-2-yl)methyl)-1H-tetrazol-5- yl)quinazoline |
| VII | 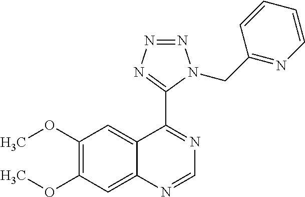 | 6,7-dimethoxy-4-(1-(pyridin-2- ylmethyl)-1H-tetrazol-5-yl)- quinazoline |

i) 6,7-Dimethoxy quinazoline derivatives:

| Compound | | |
|---|---|---|
| Number | Structure | Chemical name |
| IV b | 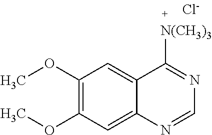 | 6,7-dimethoxy-4- quinazolinyl)- trimethylammonium chloride |
| IV d | 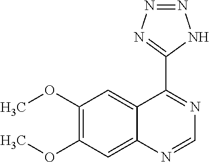 | 6,7-dimethoxy-4- (1H-tetrazol-5- yl)quinazoline |

ii) 6,7-Diethoxy quinazoline derivatives:

| Compound | | |
|---|---|---|
| Number | Structure | Chemical name |
| VIII a | 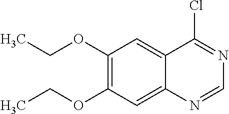 | 4-chloro-6,7-diethoxy- quinazoline |
| VIIIb | 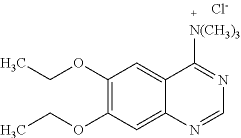 | 6,7-diethoxy-4-quinazolinyl)- trimethylammonium chloride |
| VIII c | 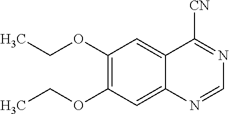 | 4-cyano-6,7-diethoxy- quinazoline |
| VIII d | 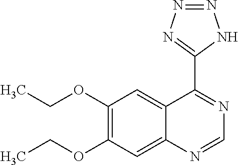 | 6,7-diethoxy-4-(1H-tetrazol-5-yl) quinazoline |

iii) 6,7-Di-n-propoxy quinazoline derivatives:

| Compound | | |
|---|---|---|
| Number | Structure | Chemical name |
| XII a | 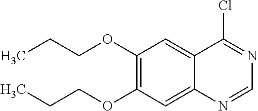 | 4-chloro-6,7-dipropoxy- quinazoline |
| XII b | 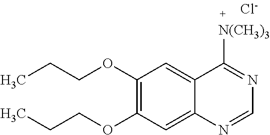 | 6,7-dipropoxy-4-quinazolinyl)- trimethylammonium chloride |
| XII c | 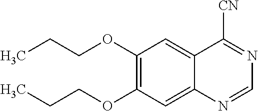 | 4-cyano-6,7-dipropoxy- quinazoline |
| XII d | 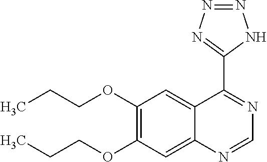 | 6,7-dipropoxy-4-(1H-tetrazol-5- yl)quinazoline |

iv) 6,7-Diethoxy substituted 4-(tetrazole-5-yl)-quinazoline derivatives of formula VIII to XI:

| Compound | | |
|---|---|---|
| Number | Structure | Chemical name |
| VIII | 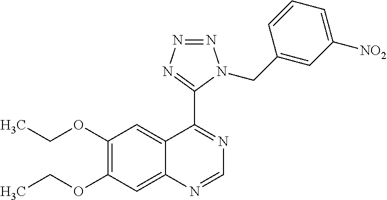 | 6,7-Diethoxy-4-(1-(3-nitrobenzyl)- 1H-tetrazol-5-yl)quinazoline |
| IX | 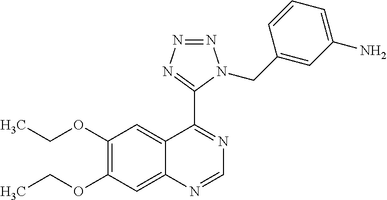 | 3-((5-(6,7-diethoxyquinazolin-4- yl)-1H-tetrazol-1-yl)methyl) aniline |
| X | 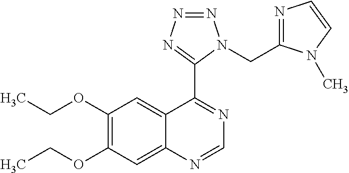 | 6,7-diethoxy-4-(1-((1-methyl-1H- imidazol-2-yl)methyl)-1H- tetrazol-5-yl)quinazoline |
| XI | 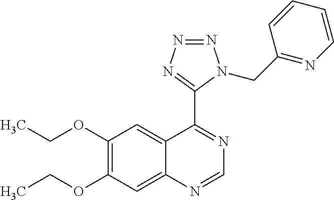 | 6,7-diethoxy-4-(1-(pyridin-2- ylmethyl)-1H-tetrazol-5- yl)quinazoline |

v) 6,7-Di-n-propoxy substituted 4-(tetrazoly-5-yl)quinazoline derivatives of formula-XII to XV:

| Compound | | |
|---|---|---|
| Number | Structure | Chemical name |
| XII | 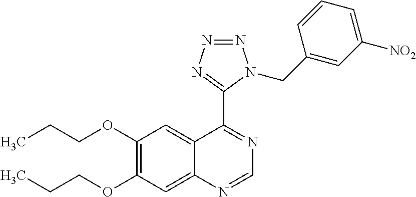 | 6,7-Di-n-propoxy-4-(1-(3- nitrobenzyl)-1H-tetrazol-5- yl)quinazoline |
| XIII | 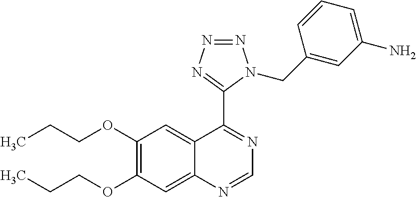 | 3-((5-(6,7-Di-n- propoxyquinazolin-4-yl)-1H- tetrazol-1-yl)methyl)aniline |
| XIV | 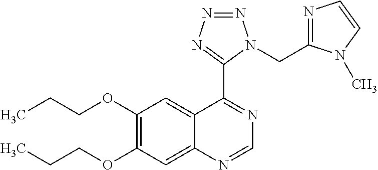 | 4-(1-((1-methyl-1H-imidazol- 2-yl)methyl)-1H-tetrazol-5- yl)-6,7-di-n- propoxyquinazoline |
| XV | 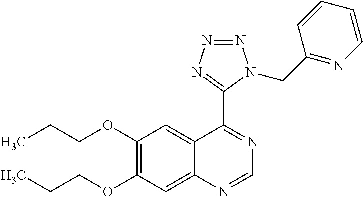 | 6,7-Di-n-propoxy-4-(1- (pyridin-2-ylmethyl)-1H- tetrazol-5-yl)quinazoline |

Within the present invention it is to be understood that, insofar as certain of the compounds of the formula I may exist in optically active or racemic forms by virtue of one or more substituents containing an asymmetric carbon atom, the invention encompasses any such optically active or racemic form which possesses anti-proliferative activity. The synthesis of optically active forms may be carried out by standard techniques of organic chemistry well known in the art, for example by synthesis from optically active starting materials or by resolution of a racemic form.

In vitro Studies

MTT Proliferation Assay

MTT [3-(4,5-dimethylthiazol-2-yl)-2,5-diphenyltetrazolium bromide]assay, first described by Mosmann in 1983, is based on the ability of a mitochondrial dehydrogenase enzyme from viable cells to cleave the tetrazolium rings of the pale yellow MTT and form dark blue formazan crystals largely impermeable to cell membranes, thus resulting in its accumulation within healthy cells. Solubilization of the cells by the addition of a detergent results in the liberation of the crystals, which are solubilized. The number of surviving cells is directly proportional to the level of the formazan product created. The color can then be quantified using a simple colorimetric assay. This assay was done using 0-1000 ng/ml concentrations of Erlotinib and its derivatives in A549 and H1299 cells. The protocol was based on ATCC and as per manufacturers instructions (Catalog Number 30-1010K).

Western Blot Analysis

Ideal drug concentrations determined from the MTT proliferation assay were used to treat 1×106 A549 or H1299 cells in appropriate media for 72 h following which cell lysates were extracted and fractionated on a 10% SDS PAGE gel under reducing conditions. The gels were blotted onto treated nylon membranes (Bio-Rad) and immunoprobed for EGFR, PI3K and AKT. The in vitro invasiveness of H1299 or A549 cells in the presence of various concentrations of NRC compounds (as determined by MTT assay) was assessed using a modified Boyden chamber assay. Cells were treated with these compounds for 48 h. 1×106 cells were suspended in 600 μl of serum-free medium supplemented with 0.2% BSA and placed in the upper compartment of the transwell chambers (Corning Costar Fischer Scientific Cat #07-200-158, Pittsburgh Pa.) coated with Matrigel (0.7 mg/ml). The lower compartment of the chamber was filled with 200 μl of serum-medium and the cells were allowed to migrate for 24 h. After incubation, the cells were fixed and stained with Hema-3 and quantified as previously described (Mohanam, et al. 1993). The migrated cells were quantified as percent invasion.

In vitro Angiogenic Assay

To determine the anti-angiogenic properties of Erlotinib and its derivatives, ideal concentration of drugs were used to treat A549 cells for 72 h as described earlier, after which, complete media was replaced with serum-free media for 12 h. This serum-free media was termed as conditioned media and used for angiogenic induction on HMEC cells grown to 80% confluency as per standard protocols.

Processes, when used to prepare the quinazoline derivative of the invention, or a pharmaceutically-acceptable salt thereof, are provided as a further feature of the invention and are illustrated by the following representative examples. Necessary starting materials may be obtained by standard procedures of organic chemistry. The preparation of such starting materials is described within the accompanying non-limiting Examples. Alternatively necessary starting materials are obtainable by analogous procedures to those illustrated, which are within the ordinary skill of an organic chemist.

The invention will be more fully described in conjunction with the following specific examples, which are not to be construed as limiting the scope of the invention.

Experimental Procedure

### EXAMPLE-1

### 1a) Preparation of 4-chloro-6, 7-dimethoxy-quinazoline

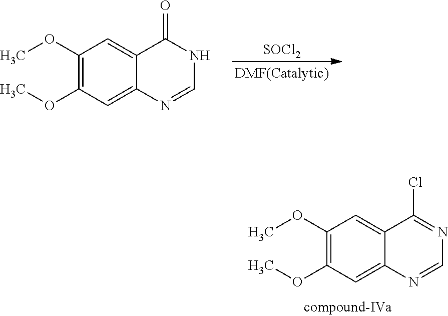

720.0 g (6.05 mol) of thionyl chloride and 50.0 g(0.243 mol) of 6,7-dimethoxy-3H-quinazoline-4-one were charged into a 2.0 L 4 necked round bottom flask connected to a mechanical stirrer, thermometer socket and double surface reflux condenser. Reaction mass temperature was raised to reflux temperature (78-80° C.). 20.0 ml of dimethyl formamide was added slowly at reflux temperature. Maintained the mass temperature at reflux for 7-8 hours under stirring. Distilled off thionyl chloride completely under vacuum at below 70° C. Cooled the mass temperature to 40° C. to 45° C. under nitrogen atmosphere 1000.0 ml of hexane was charged under stirring. Maintained the mass temperature at 40° C. to 45° C. for 30-45 min. Cooled the mass temperature 25-30° C. Maintained the mass temperature at 25-30° C. for 45-60 min under nitrogen atmosphere. Filtered the solid under nitrogen atmosphere. Solid was washed with 250.0 ml of hexane. Compound was dried in vacuum tray drier containing phosphorus pentoxide at 30-35° C. till the loss on drying is not more than 0.50% w/w. Obtained 52.50 g (yield is 96.33% by theory) of yellow colored product.

Melting range 214-220° C.

HPLC purity 96.5%.

Spectral data: FT-IR (KBr): 3060, 3041, 2951, 2838, 1618, 1562, 1505, 1429, 1360, 1336, 1232, 1163, 966, 878, 853, 806, 656, 615,493,471.

¹HNMR(DMSO-d6):. δ Value(ppm):3.89-3.91(m)2(O—CH3)(6H), 7.37(s)Ar-Ha(1H), 7.46(s)Ar-Hb91H), 9.01(s) Hc (1H).

¹³CNMR: δ value (ppm): 56.55(2C), 101.69(1C), 105.95(1C), 113.39(1C), 134.28(1C), 148.01(1C), 150.15(1C), 155.68(1C), 157.30(1C), 157.80(1C)

Mass : 225.6[M+1],224.6[M]

### 1b) Preparation of 6,7-dimethoxy-4-quinazolinyl)-trimethylammonium chloride

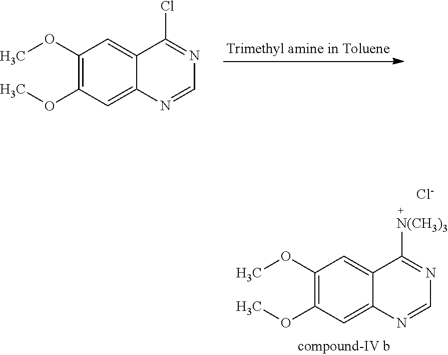

Experimental Procedure: 6.50 Lt's of trimethylamine in toluene solution was taken into a 10.0 L 4 necked round bottom flask connected to a mechanical stirrer, thermometer socket and condenser. Cooled the mass to 15-20° C. 50.0 g (0.22 mol) of 4-chloro-6,7-dimethoxy-quinazoline was charged under stirring at 15-20° C. Stirred the mass for 60-90min at 15-20° C. Insoluble compound was filtered and filtrate was collected into a 10.0 L 4 necked round bottom flask. Closed the flask with stoppers. Solution was stored at 25-35° C. for 7 days without stirring. Filtered the solid and solid was washed with 100.0 ml of toluene under nitrogen atmosphere. Compound was dried in vacuum tray drier containing phosphorus pentoxide at 30-35° C. till the loss on drying is not more than 1.0% w/w. Obtained 38.80 g(yield is 61.45% by theory) of light yellow colored product.

Melting range 218-224° C.

HPLC purity 94.8%.

Spectral data: FT-IR (KBr): 3416,3027,1615,1509,1479,1447,1413, 1361,1350, 1276, 1239, 1205,1168, 975, 884, 830,662, 572.

¹H NMR (DMSO-d6):. δ Value(ppm): 2.27(s)N—(CH3)₃(9H),3.83 (s)2(O—CH3)(6H), 7.24(s)Ar-Ha(1H),7.41(s)Ar-Hb(1H),8.49(Hc) (1H).

¹³CNMR δ value (ppm):51.1(3C),56.1(2C), 103.5(1C),108.9(1C),119.2(1C),148.1(1C), 152.3(1C),154.9(1C),159.2(1C),178.1(1C)

Mass: 284.5[M+1], 283.4[M]

### 1c) Preparation of 4-cyano -6,7-dimethoxy-quinazoline

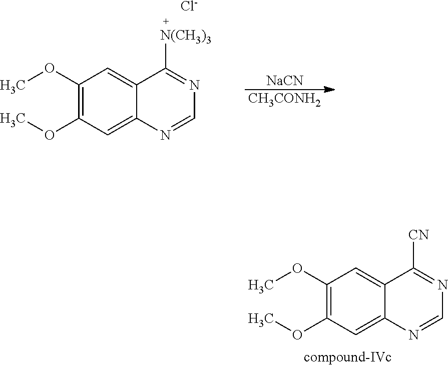

Experimental Procedure: 1800.0 ml of toluene and 37.0 g (0.13 mol) of 6,7-dimethoxy-4-quinazolinyl)-trimethylammonium chloride were charged into a 3.0 L 4 necked round bottom flask, connected to a mechanical stirrer, thermometer socket, condenser, and dean-stark apparatus. Raised the mass temperature under azeotropic conditions to reflux temperature. Maintained the mass temperature at reflux till theoretical quantity of water is separated. After water separation was completed, distilled 400.0 ml toluene. Cooled the mass temperature to 95-100° C. 46.0 g (0.78 mol) of acetamide was charged at 95-100° C. Maintained the mass temperature at 95-100° C. for 20-30 min. 19.80 g (0.40 mol) of sodium cyanide was charged at 95-100° C. Maintained the mass temperature at 95-100° C. for 20-30 min. Reaction mass temperature was raised to reflux temperature under azeotropic conditions. Maintained the mass temperature at reflux till the completion of water separation by azetropically. After water separation was completed, cooled the mass temperature to 95-100° C. Maintained the mass temperature at 90-95° C. for 6-7 hours under nitrogen atmosphere. Cooled the mass temperature to 25-30° C. 200.0 ml of DM water was charged. Stirred the mass for 20-30 min, and settled the mass for 15-20 min. Separated the top organic layer and kept aside. Charged the aqueous layer into a extraction flask. Compound was extracted with 3x300 ml of toluene. Combined the total organic layers were charged into a conical flask. Organic layer was dried with 50 g of sodium sulphate. Charged 10.0 g of activated carbon. Raised the mass temperature to 50-55° C. Maintained the mass temperature at 50-55° C. for 30-45 min. Filtered the sodium sulphate and carbon through hyflow bed and washed the sodium sulphate and carbon with 250.0 ml of toluene. Filtrate was charged into a flask and distilled off toluene completely under high vacuum, at mass temperature not crossing 65° C. Cooled the mass temperature to 25-30° C. 100.0 ml of isopropyl ether was charged. Stirred the mass temperature at 25-30° C. for 45-60 min. Filtered the solid and solid was washed with 25.0 ml of isopropyl ether. Compound was dried at 50-55° C. Obtained 22.40 g (79.85% of yield by theory) of light yellow colored product.

Melting range: 218.1° C.-219.2° C.

HPLC purity 96.5%.

Spectral data : FT-IR (KBr):3408,2927, 2233, 1614,1578, 1549, 1502, 1357, 1290, 1230, 1175, 981, 882, 843, 822, 663, 569, 494.

¹H NMR (DMSO-d6) δ value(ppm): 4.04(s)2(O—CH3)(6H), 7.30(s) Ar-Ha(1H), 7.51(s)Ar-Hb, 9.23(s)Ar-Hc(1H)

¹³CNMR δ Value (ppm): 56.70(2C), 100.88(1C), 106.67(1C), 114.92(1C), 120.82(1C), 137.61(1C), 148.83(1C), 152.57(1C), 153.0(1C), 157.62(1C).

Mass: 217.22[M+2], 216.21[M+1]

### 1d) Preparation of 6,7-dimethoxy-4-(1H-tetrazol-5-yl)quinazoline

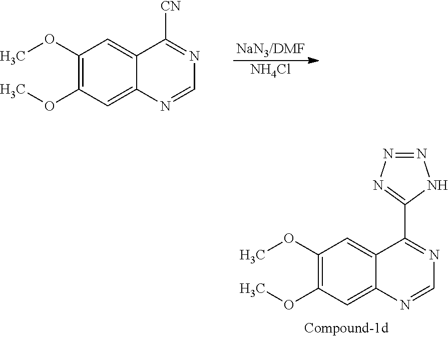

Experimental Procedure: 400.0 ml of dimethyl formamide and 20.0 g (0.09 mol) of 4-cyano-6,7-dimethoxy-quinazoline were charged into a 1.0 L 4necked round bottom flask, connected to a mechanical stirrer, thermometer socket and condenser under nitrogen atmosphere. 6.80 g (0.10 mol) of Sodium azide and 5.50 g (0.10 mol) of ammonium chloride were charged at 25-35° C. Stirred the mass for 15-20 min at 25-35° C. Stirred the mass for 15-20 min at 25-35° C. Reaction mass temperature was raised to 110-115° C. Maintained the mass temperature at 110-115° C. for 8-9 hours. Inorganic solid was filtered at 110-115° C. and the filtrate was collected into a conical flask. Cooled the filtrate to 25-30° C. 4000.0 ml of ethyl acetate was charged into a 5.0 L 4-necked round bottom flask, connected to a mechanical stirrer, thermometer socket and addition flask. Reaction mass of dimethyl formamide solution was added to ethyl acetate solution under stirring. Maintained the mass temperature at 25-30° C. for 60-90 min. Cooled the mass temperature to 0-5° C. Maintained the mass temperature at 0-5° C. for 150-180 min. Filtered the solid and solid was washed with 100.0 ml of ethyl acetate. Compound was dried at 25-30° C. under vacuum. Obtained 14.20g (yield is 59.16% by theory) of product Melting range 207.2° C.

HPLC purity: 98.6%.

Spectral data: FT-IR (KBr): 3421, 2986, 1615, 1552,1507, 1478, 1431, 1342, 1242, 998, 965, 799, 659, 450

¹H NMR(DMSO-d6) δ value(ppm): 3.92(s) 2(O—CH3)(6H), 7.34(s)Ar-Ha(1H), 8.20(broad)NH(1H), 8.97(s)Ar-Hb(1H), 9.07(s)Hc(1H)

¹³CNMR δ value (ppm): 56.36(2C), 103.28(1C), 106.72(1C), 117.39(1C), 146.81(1C), 149.87(1C), 151.43(1C), 152.58(1C), 154.65(1C), 156.54(1C).

Mass: 258.14[M], 257.18[M−1]

### I. Preparation of 6,7-Dimethoxy-4-(1-(3-nitrobenzyl)-1H-tetrazole-5yl)quinazoline (Compound-IV)

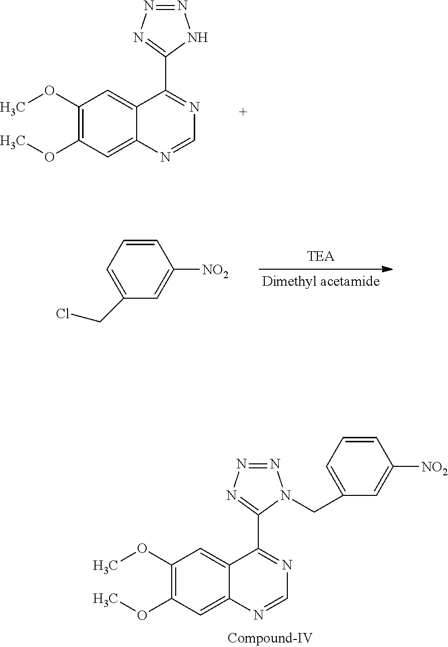

Experimental procedure: 150.0 ml of N,N-dimethyl acetamide and 10.0 g (0.038 mol) of 6,7-dimethoxy-4-(1H-tetrazol-5-yl)quinazoline were charged into a 500 ml of a 4-necked round bottom flask, connected to a mechanical stirrer, thermometer socket, condenser and addition flask under mild nitrogen atmosphere. 6.0 g (0.06 mol) of triethyl amine was added at 25-30° C. Stirred the mass for 15-20 min at 25-30° C. Reaction mass temperature was raised to 50-55° C. Maintained the mass temperature at 50-55° C. for 15-20 min. 3-Nitro benzyl chloride solution {4.50 g (0.026 mol) of 3-nitro benzyl chloride was dissolved in 37.50 ml of N,N-dimethyl acetamide} was added slowly at 50-55° C. over a period of 30-45 min. Maintained the mass temperature at 50-55° C. for 15-20 min. Raised the mass temperature to 80-85° C. Maintained the mass temperature at 80-85° C. for 7-8 hours. Cooled the mass temperature to 25-30° C. 1875.0 ml of methanol was charged into a 3.0 L 4 necked round bottom flask, connected to a mechanical stirrer, thermometer socket, condenser and addition flask at 25-30° C. Reaction mass of dimethyl acetamide solution was added to methanol solution at 25-30° C. during 60-90 min under stirring. Maintained the mass temperature at 25-30° C. for 60-90 min. Cooled the mass temperature to 0-5° C. Maintained the mass temperature at 0-5° C. for 150-180 min. Solid was filtered solid, washed with 50.0 ml of methanol. Compound was dried at 25-30° C. 11.80 g (yield 77.6% by theory) of dried compound-I is obtained.

Melting range 221.2° C.-222.2° C.

HPLC purity: 97.24%.

Spectral data: FT-IR (KBr): 3428, 3105, 2940, 1615, 1519, 1504, 1427, 1359, 1324, 1241, 1151, 1118, 1001, 966, 868, 851, 728, 658, 631, 561, 470.

¹H NMR (DMSO-d6) δ value(ppm): 3.92(s) 2(O—CH3)(6H), 6.22(s)(CH2)(2H), 7.51(s)Ar-Ha(1H), 7.64-7.7.67(t)Ar-Hb(1H), 7.87-7.89(d)(1H), 8.14-8.18(t)Ar-He(1H), 8.32(s)Ar—Hf(1H), 9.28(s)Hg(1H).

¹³CNMR δ value(ppm):51.56 (1C), 56.45(2C),103.27(1C), 106.80(1C), 118.51(1C),123.16(1C), 123.32(1C), 130.23(1C), 135.15(1C), 136.92(1C), 147.55(1C), 147.73,(1C), 149.63(1C), 151.10(1C), 151.40(1C), 152.21(1C), 156.68(1C).

Mass: 395.2[M+2],394.2[M+1]

### EXAMPLE-2

### 2. Preparation of 6,7-Dimethoxy-4-(1-(3-aminobenzyl)-1H-tetrazol-5-yl)quinazoline (Compound-V)

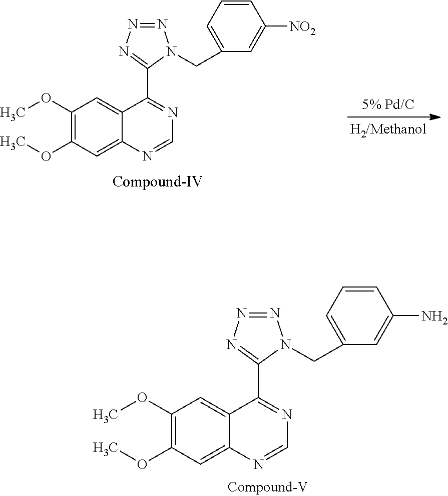

Experimental Procedure: 400.0 ml dimethyl formamide and 10.0 g (0.025 mol) of 6,7-dimethoxy-4-(1-(3-nitrobenzyl)-1H-tetrazol-5-yl)quinazoline suspension was charged into a 1.0 L hydrogenator kettle at 25-30° C. 5.0 g of 5% palladium carbon (50% wet) was charged under nitrogen atmosphere. Hydrogenation was carried at 35-40 psi under oscillation at 25-30° C. Maintained the Hydrogen gas pressure (35-40 psi) till the Hydrogen gas uptake is stopped. Filtered the catalyst through hyflow bed under nitrogen atmosphere. The catalyst was washed with 50.0 ml of dimethyl formamide under nitrogen atmosphere. Filtrate was collected into a single neck RB flask, and distilled off dimethyl formamide completely under high vacuum at below 60° C. Cooled the mass temperature to 25-30° C. and released the vacuum, 50.0 ml of hexane was charged and stirred the mass for 45-60 min at 25-30° C. Filtered the solid, washed with 25.0 ml of hexane. Compound was dried at 25-30° C. 8.40 g of crude product is obtained. The crude product was purified by column chromatography in a silica column using mobile phase as ethylacetate and hexane mixture. Obtained 5.20 g (56.3% yield by theory) of product with HPLC purity of 99.3%.

Spectral data: FT-IR (KBr):3430,3008, 2930, 1613, 1551, 1501, 1429, 1375, 1320, 1236, 1150, 1001,963, 871, 845, 797, 775,693, 657, 627, 446.

¹H NMR(DMSO-d6) δ value(ppm): 3.92(s)(O—CH3) (3H),4.03(s)(O—CH3) (3H), 5.07(s)CH2(2H), 5.95-5.98(s) NH2(2H), 6.34-6.43(m)Ar-Ha,Hb,Hc(3H), 6.86-6.89(t)Ar-Hd(1H),7.50(s)Ar—He(1H), 8.04(s)Ar—Hf(1H), 9.31(s)Hg(1H)

¹³CNMR δ value(ppm): 51.56 (1C), 56.45(2C),103.07(1C), 106.80(1C), 112.91(1C), 113.61(1C), 115.07(1C), 118.49(1C), 129.08(1C), 129.08(1C), 135.31(1C), 147.69(1C), 148.95(1C), 150.63(1C), 151.42(1C), 152.31(1C), 156.76(1C).

Mass: 365.3[M+2],364.3[M+1]

### II. HCl: Preparation of 6,7-Dimethoxy-4-(1-(3-aminobenzyl)-1H-tetrazol-5-yl)quinazoline hydrochloride salt

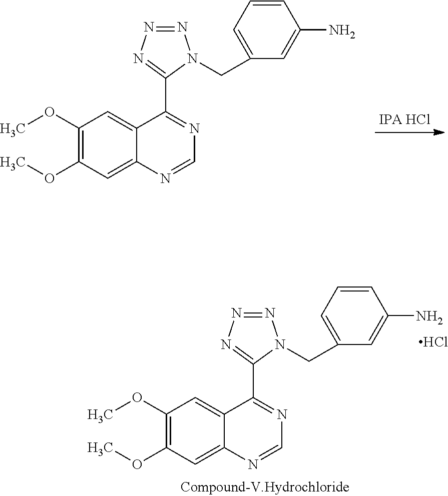

Experimental Procedure: Charged 200.0 ml of methylene chloride and 5.0 g (0.013 mol) of 6,7-Dimethoxy-4-(1-(3-aminobenzyl)-1H-tetrazol-5-yl)quinazoline into a 500 ml of 4necked round bottom flask, connected to a mechanical stirrer, thermo meter socket and condenser at 25-30° C. Stirred the mass for 15 min. After dissolution is clear, 6.0 g of IPA HCl was added. Stirred the mass for 1 hour. Methylene chloride was distilled of up to remaining the total mass volume 30.0 ml. 200 ml of hexane was added. Stirred the mass for 1 hour. Filtered the solid and solid was washed with 30.0 ml of hexane. Compound was dried at 55-60° C. Obtained light yellow colored dry compound 4.80 g (yield is 87.2% by theory).

Melting range 234.8-236.3° C. Product purity: 99.5% by HPLC.

Spectral data: FT-IR (KBr): 3424, 3227, 3094, 3052, 2978, 2878, 2746, 1665, 1595, 1508, 1471, 1435, 1411, 1352, 1312, 1286, 1260, 1239, 1205, 1131, 1110, 1065, 1050, 917, 885, 854, 827, 778, 721, 684, 534, 476.

¹H NMR(DMSO-d6) δ value(ppm): 3.94(s)(O—CH3) (3H),4.03(s)(O—CH3) (3H), 5.07(s)CH2(2H), 6.09(s) NH2(2H), 6.34-6.43(m)Ar-Ha,Hb,Hc(3H), 6.86-6.89(t)Ar-Hd(1H),7.50(s)Ar—He(1H), 8.04(s)Ar—Hf(1H), 9.31(s)Hg(1H)

¹³CNMR δ value(ppm):51.95(1C), 56.43(1C), 103.26(1C), 106.75(1C), 118.35(1C), 122.77(1C), 127.54(1C), 130.01(1C), 132.79(1C), 136.68(1C), 147.68(1C), 150.9391C), 151.37(1C), 152.28(1C), 156.60(1C).

Mass : 400.3[M+1], 398.3[M−1].

### EXAMPLE-3

### Preparation of 6,7-dimethoxy-4-[1-(1-methyl-1H-imidazol-2-ylmethyl)-1H-tetrazol-5-yl]-quinazoline (Formula-VI)

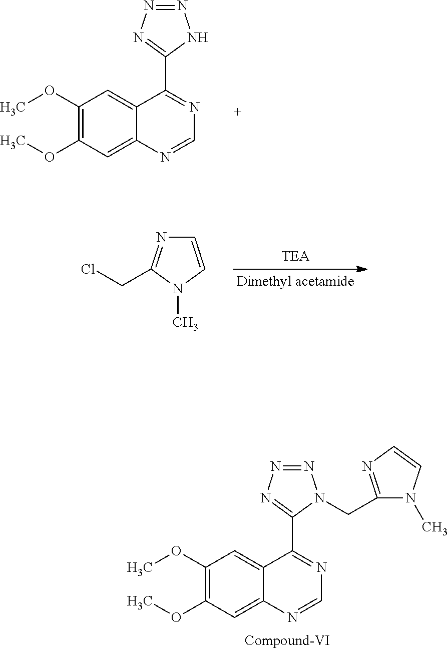

Experimental procedure: 50.0 ml of N,N-dimethyl acetamide and 5.0 g (0.019 mol)of 6,7-dimethoxy-4-(1H-tetrazol-5-yl)quinazoline were charged into a 250 ml 0f 4necked round bottom flask, connected to a mechanical stirrer, thermometer socket, condenser and addition flask under mild nitrogen atmosphere. 3.80 g (0.038 mol) of triethyl amine was added at 25-30° C. Stirred the mass for 15-20 min at 25-30° C. Reaction mass temperature was raised to 50-55° C. Maintained the mass temperature at 50-55° C. for 15-20 min. 2-chloro methyl-1-methyl-imidazloe solution[2.50 g(0.019 mol) 2-chloro methyl-1-methyl-imidazloe was dissolved in 25.0 ml of N,N-dimethyl acetamide] was added slowly at 50-55° C. for 30-45 min. Maintained the mass temperature at 50-55° C. for 15-20 min. Raised the mass temperature to 80-85° C. Maintained the mass temperature at 80-85° C. for 7-8 hours. N,N-dimethyl acetamide was completely distilled under vacuum. Crude compound was purified by the column chromatography by using hexane and ethyl acetate. Obtained pure compound weight 2.40 g (yield 35.2% by theory).

Spectral data: Mass : 353 [M+1], 352.0[M]

### EXAMPLE-4

### Preparation of 6,7-dimethoxy-4-[1-(pyridine-2ylmethyl) -1H-tetrazol-5-yl]-quinazoline

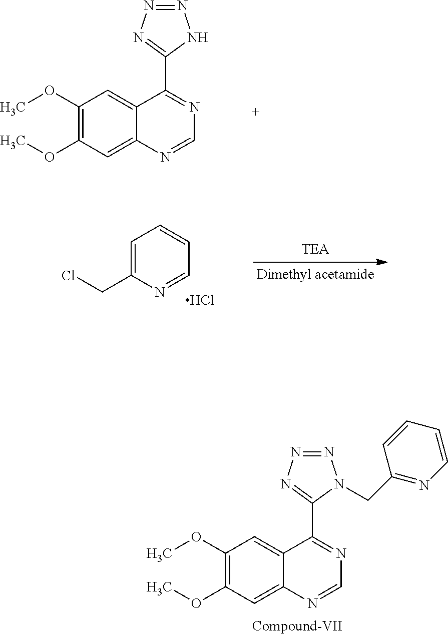

Experimental procedure: 50.0 ml of N,N-dimethyl acetamide and 5.0 g (0.019 mol) of 6,7-dimethoxy-4-(1H-tetrazol-5-yl)quinazoline were charged into a 250 ml of 4 necked round bottom flask, connected to a mechanical stirrer, thermometer socket, condenser and addition flask under mild nitrogen atmosphere. 3.80 g (0.038 mol) of triethyl amine was added at 25-30° C. Stirred the mass for 15-20 min at 25-30° C. Reaction mass temperature was raised to 50-55° C. Maintained the mass temperature at 50-55° C. for 15-20 min. 2-chloro methyl pyridine hydrochloride solution [3.20 g (0.019 mol) 2-chloro methyl pyridine hydrochloride was dissolved in 25.0 ml of N,N-dimethyl acetamide] was added slowly at 50-55° C. for 30-45 min. Maintained the mass temperature at 50-55° C. for 15-20 min. Raised the mass temperature to 80-85° C. Maintained the mass temperature at 80-85° C. for 7-8 hours. N,N-dimethyl acetamide was completely distilled off under vacuum. Crude compound was purified by the column chromatography by using hexane and ethyl acetate. Obtained 1.90 g (yield 28.0% by theory) of pure compound weight.

Spectral data: Mass: 350 [M+1], 349.0[M]

### EXAMPLE-5 TO 8

The analogous compounds of 3,4-diethoxy derivatives of quinazoline compounds VIII to XI and the their intermediates VIII a to VIII d are prepared as per the procedure mentioned in examples-1a to 1d and IV to VII

i) Mass spectral properties of compounds VIII a to VIII d

| Compound | Molecular | Molecular | Mass peaks | |
|---|---|---|---|---|
| Number | formula | weight | Peak-i | Peak-ii |
| VIII a | C₁₂H₁₃N₂O₂Cl | 252.5 | 253.7[M + 1] | 252.5[M] |
| VIII b | C₁₅H₂₂N₃O₂Cl | 311.5 | 312.6[M + 1] | 311.6[M] |
| VIII c | C₁₃H₁₃N₃O₂ | 243.0 | 245.2[M + 2] | 244.2[M + 1] |
| VIII d | C₁₃H₁₄N₆O₂ | 286.0 | 286.3[M] | 285.1[M − 1] |

ii) Mass spectral properties of compounds VIII to XI

| Compound | Molecular | Molecular | Mass peaks | |
|---|---|---|---|---|
| Number | formula | weight | [M + 2] | [M + 2] |
| VIII | C₂₀H₁₉N₇O₄ | 421.0 | 423.4 | 422.4 |
| IX | C₂₀H₂₁N₇O₂ | 391.0 | 393.4 | 392.4 |
| X | C₁₈H₂₀N₈O₂ | 380.0 | 382.4 | 381.4 |
| XI | C₁₉H₁₉N₇O₂ | 377.0 | 379.4 | 378.4 |

### EXAMPLE-9 TO 12

The analogous compounds of 3,4-dipropoxy derivatives of quinazoline compounds XII to XV and the their intermediates XIIa to XIId are prepared as per the procedure mentioned in examples-1a to 1d and IV to VII

iii) Mass spectral properties of compounds XII a to XII d

| Compound | Molecular | Molecular | Mass peaks | |
|---|---|---|---|---|
| Number | formula | weight | Peak-i | Peak-ii |
| XII a | C₁₄H₁₇N₂O₂Cl | 280.5 | 281.7 [M + 1] | 280.7 [M] |
| XII b | C₁₇H₂₆N₃O₂Cl | 339.5 | 340.6 [M + 1] | 339.6 [M] |
| XII c | C₁₅H₁₇N₃O₂ | 271.0 | 273.2 [M + 2] | 272.2 [M + 1] |
| XII d | C₁₅H₁₈N₆O₂ | 314.0 | 314.3 [M] | 313.1 [M − 1] |

iv) Mass spectral properties of compounds XII to XV

| Compound | Molecular | Molecular | Mass peaks | |
|---|---|---|---|---|
| Number | formula | weight | [M + 2] | [M + 1] |
| XII | C₂₂H₂₃N₇O₄ | 449.0 | 451.4 | 450.4 |
| XIII | C₂₂H₂₅N₇O₂ | 419.0 | 421.4 | 420.4 |
| XIV | C₂₀H₂₄N₈O₂ | 408.0 | 410.4 | 409.4 |
| XV | C₂₁H₂₃N₇O₂ | 405.0 | 407.4 | 406.4 |

### EXAMPLE 13

### In vitro Kinase Profiling of NRC-1005 (Compound V)

Kinase assays: Assays were performed as described in Fabian et al. (2005) Nature Biotechnology, vol. 23, p. 329. Kinase-tagged T7 phage strains were grown in parallel in 24 or 96-well blocks in an E. coli host derived from the BL21 strain. E. coli were grown to log-phase and infected with T7 phage from a frozen stock (multiplicity of infection ˜0.1) and incubated with shaking at 32° C. until lysed (˜90 minutes). The lysates were centrifuged and filtered to remove cell debris. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining phage lysates, liganded affinity beads, and test compounds in 1 × binding buffer (20% SeaBlock, 0.17× PBS, 0.05% Tween 20, 6 mM DTT).

Test compounds were prepared as 40x stocks in DMSO and diluted into the aqueous environment. 2.5% DMSO was added to control assays lacking a test compound. All reactions were performed in polystyrene 96-well plates that had been pretreated with blocking buffer in a final volume of 0.04 ml. The assay plates were incubated at room temperature with shaking for 1 hour, long enough for binding reactions to reach equilibrium, and the affinity beads were washed four times with wash buffer (1× PBS, 0.05% Tween 20, 1 mM DTT) to remove unbound phage. After the final wash, the beads were resuspended in elution buffer (1× PBS, 0.05% Tween 20, 2 μM non-biotinylated affinity ligand) and incubated at room temperature with shaking for 30 minutes. The phage titer in the eluates was measured by quantitative PCR.

Result:

NRC-1005 (the compound V) was found to possess very high inhibiting activity against various kinases from a panel of 80 kinases by the method of KinomeScan™ of Ambit Biosciences Corp, San Diego, Calif. The kinases inhibited were CSF1R, FLT3, FLT3 (K663Q), GAK, KIT, KIT (V559D), KIT (V559D, V654A), PDGFRA, and PDGFRB.

### EXAMPLE 14

### In vitro and In vivo Evaluation and Therapeutic Efficacy

Using the approved Erlotinib as a positive control, the biological activity of compounds of the present invention were tested.

Western Blot Analysis (FIG.-1):

Test compound concentrations determined from the MTT proliferation assay were used to treat 1×10⁶ A549 or H1299 cells in appropriate media for 72 h followed by western blotting. Using A549 cells the Erlotinib and Compound V (NRC-1005) induced dose dependent decreases in EGFR expression levels. Those results show a dose dependent decrease in EGFR expression levels for Compound V (Development code NRC-1005) which is comparable to that observed for Erlotinib with a dose of Compound V which is 10% of the corresponding reference drug. In H1299 cells, no significant change in expression levels of PI3K or AKT was observed in any of drug treatments.

Matrigel Invasion Assay (FIG.-2 & 3)

The in vitro invasiveness of H1299 and A549 cells in the presence of various concentrations of Erlotinib and Compounds IV (NRC-1004) and V (NRC-1005) was assessed using a modified Boyden chamber assay. Cells were treated with these compounds for 48 h. 1×10⁶ cells were suspended in 600 μl of serum-free medium supplemented with 0.2% BSA and placed in the upper compartment of the transwell chambers (Corning Costar Fischer Scientific Cat #07-200-158, Pittsburgh Pa.) coated with Matrigel (0.7 mg/ml). The lower compartment of the chamber was filled with 200 μl of serum-medium and the cells were allowed to migrate for 24 h. After incubation, the cells were fixed and stained with Hema-3 and quantified as previously described (Mohanam, et al. 1993). The migrated cells were quantified as percent invasion.

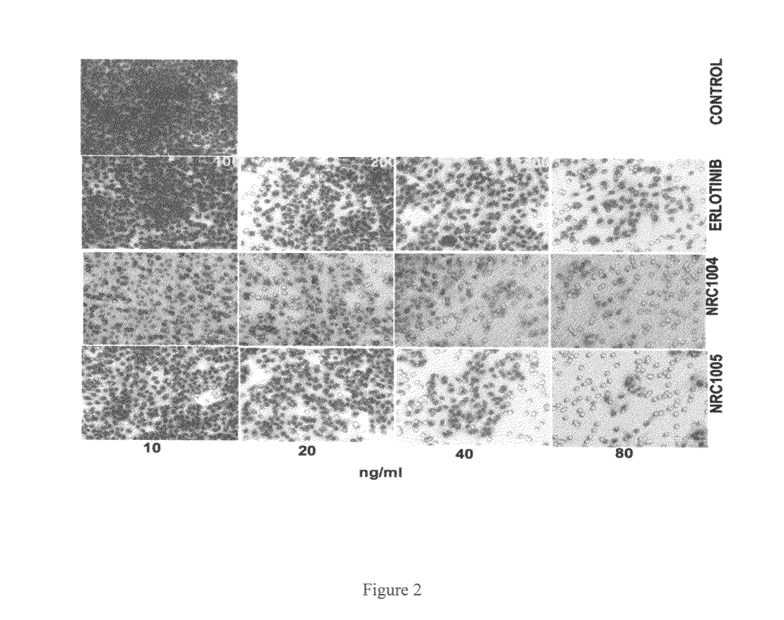

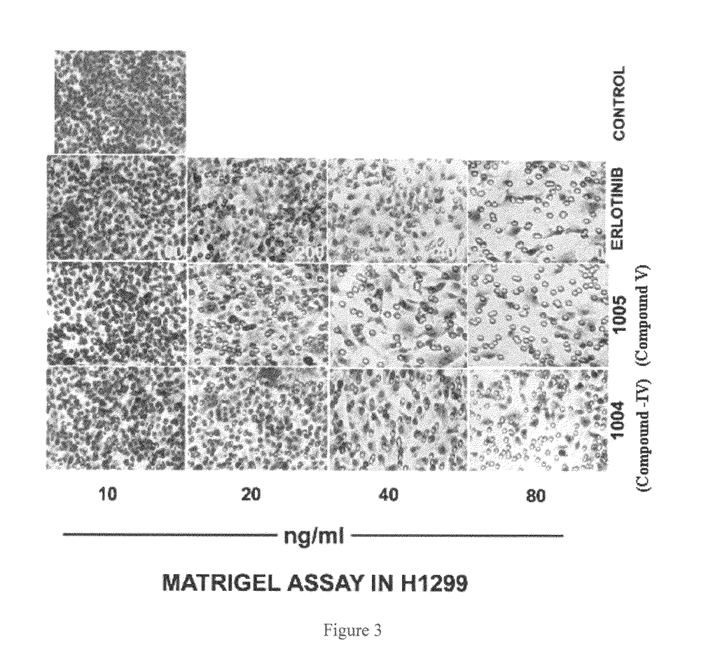

Matrigel invasion assay was performed as described in materials and methods. FIG. 2 illustrates results of a matrigel invasion assay of A549 cells treated with Erlotinib, Compound IV (NRC-1004), and Compound V (NRC-1005). Using A549 cells the control compound Erlotinib decreased invasiveness in a dose dependent manner from 100 to 800ng/ml. Compound IV (Development code NRC-1004) and Compound V (Development code NRC-1005) caused retardation of invasion similar to Erlotinib at 1/10^{th} concentration (10-80 ng/ml ). FIG. 3 illustrates results of a matrigel invasion assay of H1299 cells treated with Erlotinib, Compound IV (NRC-1004), and Compound V (NRC-1005). Using H1299 cells similar retardation patterns of invasion was observed.

In Vivo Evaluation on Subcutaneous Lung Tumors in Nude Mice (FIG.-4)

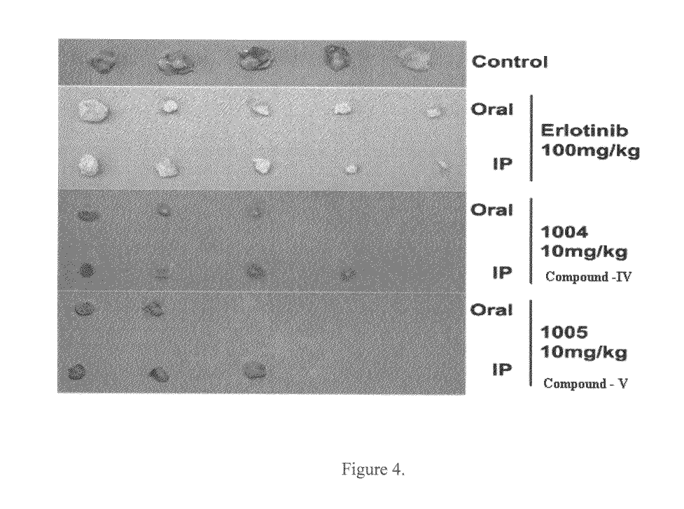

Nude mice were implanted with 2×10⁶ A549 cells in the right hind limb flank. Upon the observed formation of a tumor (>2 mm), mice were given oral or ip treatments of Erlotinib, and above-mentioned compounds at 1/10^{th} of dose of Erlotinib. From a literature search, 100 mg/kg of Erlotinib had been identified as the base line dose. FIG. 4 illustrates the decrease in tumor size induced by oral administration of Erlotinib, Compound IV (NRC-1004), and Compound V (NRC-1005) in nude mice implanted with A549 human lung tumors. Compounds IV (NRC-1004) and V (NRC-1005) caused retardation of tumor growth similar to Erlotinib at 1/10^{th} concentration (10-80 ng/ml).

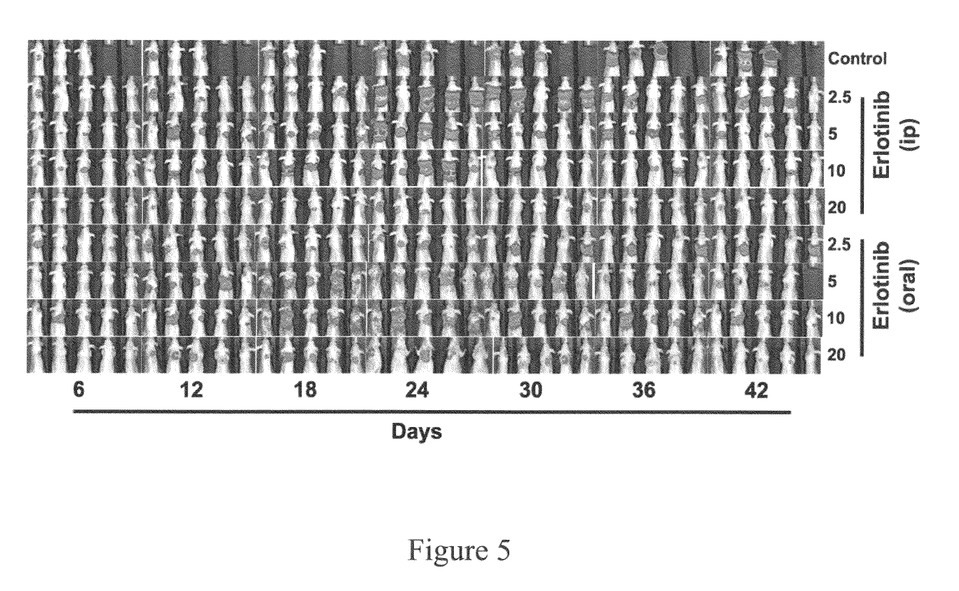

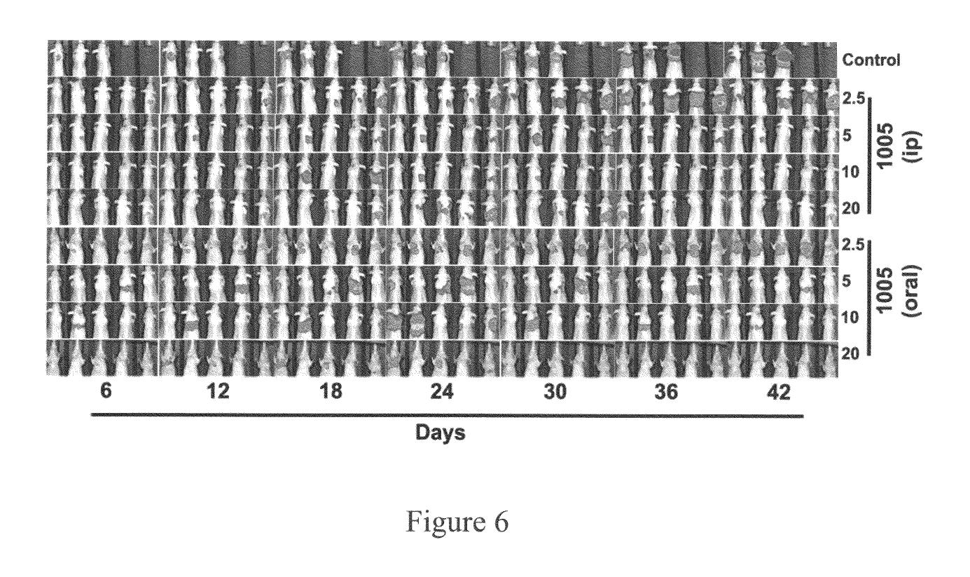

Nude Mice Implanted with A549 Luciferase Expressing Cells (FIGS. 5 and 6):

Nude mice implanted with A549 luciferase expressing cells treated with various concentrations of Erlotinib HCl and NRC-1005 (Compound V) by oral and ip routes were observed for tumors and the pictorial observations are given as FIGS. 5 and 6. FIG. 5 illustrates nude mice implanted with A549 luciferase expressing cells and then treated with various concentrations of Erlotinib by oral or ip routes. FIG. 6 illustrates nude mice implanted with A549 luciferase expressing cells and then treated with various concentrations of Compound V (NRC-1005) by oral or ip routes.

It was observed that the group treated with NRC-1005 (Compound V) fared much better in reducing the size of tumor than the group treated with Erlotinib HCl. No tumors were observed at the end of 42 days treatment with NRC-1005 (Compound V) where as residual tumors were still present in the group treated with Erlotinib HC1 both by oral and ip routes.

Curative Effect from in Vivo Studies in Nude Mice:

The curative effect as a ratio of number of animals cured to the number of animals used in the study and presented in Table 1

TABLE 1
Curative Effect of NRC-1005 (Compound V) and
Erlotinib HCl on Lung Cancer

| | Concentration | Cure |
|---|---|---|
| Drug | mg/kg | ratio |
| Erlotinib IP | 2.5 | 1/5 |
| | 5 | 2/5 |
| | 10 | 2/5 |
| | 20 | 3/5 |
| Erlotinib Oral | 2.5 | 2/5 |
| | 5 | 0/5 |
| | 10 | 1/5 |
| | 20 | 2/5 |
| NRC1005 IP | 2.5 | 1/5 |
| | 5 | 3/5 |
| | 10 | 3/5 |
| | 20 | 4/5 |
| NRC1005 Oral | 2.5 | 0/5 |
| | 5 | 2/5 |
| | 10 | 4/5 |
| | 20 | 5/5 (100%) |

It can be seen that the cure ratio is close to 100% in the case of NRC-1005(Compound V) where as the ratio is between 40-60% in the case of study group with Erlotinib HCl

Advantages of Present Invention:

1. The above-mentioned novel compounds are superior to the existing standard therapies of non-small cell lung cancers such as Gefitinib and Erlotinib and are potentially useful in lung cancer therapy.

2. The above-mentioned novel compounds are also working on other area such as pancreatic cancer and are potentially useful in pancreatic-cancer therapy.

3. The above-mentioned novel compounds are also working on other area such as throat and oral cancer and are potentially useful in throat and oral cancer therapy.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A method of treating a hyper proliferative disease in a mammal which comprises administering to said mammal a therapeutically effective amount of the compound of formula I 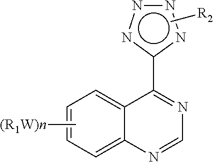
where
n is 1, 2, or 3;
W is selected from a single bond, —O—, —S—, —COR₆, —NH—, —SO—, —SO₂—, —NR₆CO—, —CONR₆—, —SO₂NR₇—, —NR₇SO₂—, or —NR₈— (wherein R₆, R₇ and R₈ each independently represents hydrogen, C₁-C₆alkyl, C₃-C₆cycloalkyl, C₂-C₅alkeynl, C₂-C₅alkynyl,
each R₁ is R₉ where R₉ is independently selected from C₁-C₆ branched alkyl, C₂-C₆ branched alkenyl or C₂-C₆ branched alkynyl;
or each R₁ is independently selected from the group consisting of hydrogen, halogen, hydroxy, amino, hydroxylamino, carboxy, nitro, guanidino, ureido, cyano, trifluoromethyl, azido;
or each R₁ is independently selected from the group consisting of C₁-C₆alkyl, C₃-C₆ cycloalkyl, aryl, heterocyclyl, R₃-substituted aryl, R₃-substituted heterocyclyl, aryl C₁-C₆alkoxy, C₃-C₆cycloalkoxy, (C₁-C₆)alkanoyloxy, R₅-aryloxy, C₁-C₆alkoxy C₁-C₆alkyloxy, C₁-C₆alkoxy-C₃-C₆cycloalkyloxy, C₁-C₆alkoxy-R₅-aryloxy, C₁-C₆alkoxy-heterocyclyloxy, C₁-C₆alkoxy-fused-heterocyclyloxy, N-mono(C₁-C₆)alkylamino, N,N-di(C₁-C₆)alkylamino, formamido, amido, acetamido, C₁-C₆-alkoxyamino, hydrazino, trifluoromethoxy, alkenyl, alkynyl, aryl, heterocyclyl, fused aryl, fused heteroaryl and fused heterocyclyl; where R₃ is selected from C₁-C₆ alkyl, C₃-C₆ cycloalkyl, aryl, and aralkyl; R₅ is independently hydrogen or R₄; and where R₄ is C₁-C₄ alkyl;
or each R₁ is independently selected from R₉-substituted by halogen, hydroxy, amino, hydroxylamino, carboxy, nitro, guanidino, ureido, cyano, trifluoromethyl, azido; wherein R₉ is selected from the group consisting of R₄, —OR₅, —NR₅R₅, —C(O)R₆, —NHOR₄, —OC(O)R₅, P and -QR₄; R₆ is R₃, —OR₅ or —NR₅R₅; P is selected from piperidino, morpholino, pyrrolidino, 4-R₃-piperazin-1-yl, imidazol-1-yl, 4-pyridon-1-yl, —(C₁-C₄alkylene)(CO₂H), phenoxy, phenyl, phenylsulfonyl, C₂-C₄alkenyl, and —(C₁-C₄alkylene)C(O)NR₅ R₅; and Q is S, SO or SO₂;
or each R₁ is independently selected from phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R₄—(C₂-C₄)-alkanoylamino and wherein said —NHSO₂ R₄, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R₄—(C₂-C₄)-alkanoylamino R₁ groups are optionally substituted by 1 or 2 substituents independently selected from halo, C₁-C₄alkyl; cyano, methanesulfonyl and C₁-C₄alkoxy;
R₂ is hydrogen or selected from the group consisting of C₁-C₆alkyl, C₃-C₆cycloalkyl, (C₁-C₆)carbonyloxyalkyl, R₄-aryl, R₄-aryl substituted with (R₁₁)m, wherein m=1, 2 or 3 and R₁₁ is independently selected from the group consisting of hydrogen, halogen, hydroxy, hydroxylamino, carboxy, nitro, guanidino, ureido, cyano, trifluoromethyl, azido, amino, methyl, or R₃ (as defined above), —R₄-fused aryl, —R₄-fused aryl substituted with (R₁₁)m, -R₄-heterocyclyl, —R₄-heterocyclyl substituted with (R₁₁)m, —R₄-fused heterocyclyl, —R₄-fused heterocyclyl substituted with (R₁₁)m, —R₄-C₁-C₆alkyloxy, —R₄-C₁-C₆alkyloxy substituted with (R₁₁)m, —R₄-C₃-C₆cycloalkyloxy, —R₄-C₃-C₆cycloalkyloxy substituted with (R₁₁)m, —C₁-C₆alkoxy-R₅-aryloxy; —C₁-C₆alkoxy-R₅-aryloxy substituted with (R₁₁)m, -C₁-C₆alkoxy hetero-cyclyloxy, —C₁-C₆alkoxy-heterocyclyloxy substituted with (R₁₁)m, C₁-C₆alkoxy fused heterocyclyloxy, C₁-C₆alkoxy fused heterocyclyloxy substituted with (R₁₁)m, N-mono(C₁-C₆)alkylamino, N-mono(C₁-C₆)alkylamino substituted with (R₁₁)m, N,N-di(C₁-C₆)alkylamino, N,N-di(C₁-C₆)alkylamino substituted with (R₁₁)m, formamido, amido, acetamido, C₁-C₆alkoxyamino, hydrazino, trifluoromethoxy, C₂-C₆ alkenyl, C₂-C₆ alkenyl, substituted with (R₁₁)m, C₂-C₆alkynyl, C₂-C₆alkynyl substituted with (R₁₁)m;
or a pharmaceutically acceptable salt thereof.

2. The method of claim 1, where in the hyper proliferative disorder is cancer

3. The method of claim 2, wherein said cancer is lung, squamous cell, bladder, gastric, pancreatic, breast, head, neck, esophageal, brain, gynecological or thyroid cancer.

4. The method of claim 1, wherein the hyperproliferative disease is noncancerous.

5. Use of a compound according to claim 1 in the manufacture of a medicament for treating the human or animal body.

6. The method of claim 1, comprising administering:
a) 6,7-Dimethoxy-4-(1-(3-nitrobenzyl)-1H-tetrazol-5-yl)quinazoline;
b) 3-((5-(6,7-dimethoxyquinazolin-4-yl)-1H-tetrazol-1-yl)methyl)aniline;
b′) 6,7-Dimethoxy-4-(1-(3-aminobenzyl)-1H-tetrazol-5-yl)quinazoline hydrochloride;
c) 6,7-dimethoxy-4-(1-((1-methyl-1H-imidazol-2-yl)methyl-1H-tetrazol-5-yl)quinazoline;
d) 6,7-dimethoxy-4-(1-(pyridin-2-ylmethyl)-1H-tetrazol-5-yl)-quinazoline;
e) 6,7-diethoxy-4-(1H-tetrazol-5-yl)quinazoline;
f) 6,7-diethoxy-4-(1-(3-nitrobenzyl)-1H-tetrazol-5-yl)quinazoline;
g) 3-((5-(6,7-diethoxyquinazolin-4-yl)-1H-tetrazol-1-yl)methyl)aniline;
h) 6,7-diethoxy-4-(1-((1-methyl-1H-imidazol-2-yl)methyl)-1H-tetrazol-5-yl)quinazoline;
i) 6,7-diethoxy-4-(1-(pyridin-2-ylmethyl)-1H-tetrazol-5-yl)quinazoline;
j) 6,7-dipropoxy-4-(1H-tetrazol-5-yl)quinazoline;
k) 6,7-di-n-propoxy-4-(1-(3-nitrobenzyl)-1H-tetrazol-5-yl)quinazoline;
l) 3-((5-(6,7-di-n-propoxyquinazolin-4-yl)-1H-tetrazol-1-yl)methyl)aniline;
m) 4-(1-((1-methyl-1H-imidazol-2-yl)methyl)-1H-tetrazol-5-yl) 6,7-di-n-propoxy quinazoline; or
n) 6,7-di-n-propoxy-4-(1-(pyridin-2-ylmethyl)-1H-tetrazol-5-yl)-quinazoline.

7. The method of claim 1, comprising administering a compound of Formula IV, Formula V or a pharmaceutically acceptable salt thereof: [chemical structure]
